(19) United States
(12) Patent Application Publication
(10) Pub. No.: US 2012/0122161 A1
(43) Pub. Date: May 17, 2012

(54) Sorting Asymmetrically Tagged Nucleic Acids by Selective Primer Extension

(76) Inventors: Esther Musgrave-Brown, Great Chesterford (GB); Gi Mikawa, Cambridge (GB); Robert Osborne, Great Chesterford (GB); Andrew Slatter, London (GB)

(21) Appl. No.: 13/321,317

(22) PCT Filed: May 21, 2010
(86) PCT No.: PCT/IB2010/001392

§ 371 Date: Jan. 26, 2012

Related U.S. Application Data
(60) Provisional application No. 61/180,583, filed on May 22, 2009.

(51) Int. Cl. C12P 19/34

(52) U.S. Cl. 435/91.21

(57) ABSTRACT

The present invention provides methods and compositions for amplifying and sorting adapter tagged nucleic acid fragments using selective primer extension. Immortalized pooled polynucleotide samples and method of producing the same are also provided.

### BACKGROUND

A major goal in genetics research is to understand how sequence variations in the genome relate to complex traits, particularly susceptibilities for common diseases such as diabetes, cancer, hypertension, and the like, e.g. Collins et al, Nature, 422: 835-847 (2003). The draft sequence of the human genome has provided a highly useful reference for assessing variation, but it is only a first step towards understanding how the estimated 10 million or more common single nucleotide polymorphisms (SNPs), and other polymorphisms, such as inversions, deletions, insertions, and the like, determine or affect states of health and disease. Many powerful analytical approaches have been developed to address this problem, but none appear to have adequate throughput or flexibility for the types of studies required to associate traits practically and reliably with genomic variation, e.g. Syvanen, Nature Reviews Genetics, 2: 930-942 (2001). For example, it would be desirable to carry out trait-association studies in which a large set of genetic markers from populations of affected and unaffected individuals are compared. Such studies depend on the non-random segregation, or linkage disequilibrium, between the genetic markers and genes involved in the trait or disease being studied. Unfortunately, the extent and distribution of linkage disequilibrium between regions of the human genome is not well understood, but it is currently believed that successful trait-association studies in humans would require the measurement of 30-50,000 markers per individual in populations of at least 300-400 affected individuals and an equal number of controls, Kruglyak and Nickerson, Nature Genetics, 27: 234-236 (2001); Lai, Genome Research, 11: 927-929 (2001); Risch and Merikangas, Science, 273: 1516-1517 (1996); Cardon and Bell, Nature Reviews Genetics, 2: 91-99 (2001).

One approach to dealing with such whole-genome studies is to create subsets of genomic DNA having reduced complexity with respect to the genomes being analyzed in order to simplify the analysis, e.g. Lisitsyn et al, Science, 259: 946-951 (1993); Vos et al, Nucleic Acids Research, 23: 4407-4414 (1995); Dong et al, Genome Research, 11: 1418-1424 (2001); Jordan et al, Proc. Natl. Acad. Sci., 99: 2942-2947 (2002); Weissman et al, U.S. Pat. No. 6,506,562; Sibson, U.S. Pat. No. 5,728,524; Degau et al, U.S. Pat. No. 5,858,656. Unfortunately, most of these techniques rely on some form of subtraction, sequence destruction, or direct or indirect size selection to create subsets, which are difficult to implement and reduce sensitivity.

In view of the above, the field of genetic analysis would be advanced by the availability of a method for converting a highly complex population of DNA, such as a mixture of genomes, into subsets having reduced complexity without requiring subtraction, or other sequence destroying, steps.

### SUMMARY

Aspects of the present invention provides methods and compositions for the production, amplification and sequence-specific sorting of tagged poly nucleotide fragments (e.g., asymmetrically tagged polynucleotides). In certain embodiments, the sequence-specific sorting employs a selective primer extension (SPE) approach. Immortalized pooled polynucleotide samples and method of producing the same are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. Indeed, the dimensions of the various features are arbitrarily expanded or reduced for clarity. Included in the drawings are the following figures:

FIGS. 1A and 1B show exemplary structural components of asymmetric adapters that find use in practicing aspects of the subject invention.

FIG. 2 shows an exemplary embodiment of producing asymmetrically tagged nucleic acid fragments according to aspects of the subject invention.

FIG. 3 shows an exemplary asymmetric adapter that finds use in aspects of the present invention.

FIG. 4 shows the adapters of FIG. 3 ligated to a nucleic acid fragment.

FIG. 5 shows first strand synthesis of the adapter ligated fragment of FIG. 4 using a biotinylated synthesis primer.

FIG. 6 shows an alternative asymmetric adapter containing a biotin moiety.

FIGS. 7 and 8 show exemplary alternative schemes for thermocycling-based linear amplification of an adapter-ligated fragment.

FIG. 9 shows an exemplary scheme to avoid unwanted amplification products in a linear amplification reaction.

FIG. 10 shows an exemplary scheme for template strand removal after amplification.

FIGS. 11 to 14 show an exemplary amplification scheme employing a NuGEN SPIA®-based amplification system.

FIGS. 15 and 16 show an exemplary scheme for sorting by Selective Primer Extension (SPE).

FIG. 17 shows an exemplary scheme for employing terminal transferase and ddNTPs in an SPE reaction.

FIGS. 18, 19 and 20 show exemplary schemes for performing multiple rounds of SPE using a NuGEN SPIA®-based system.

FIG. 21 shows production of an asymmetrically tagged nucleic acid fragment for use in an SPE sorting scheme.

FIG. 22 shows a summary of the 7 steps involved in an exemplary SPE sorting protocol.

FIGS. 23 to 27 show details of each step in the exemplary SPE sorting protocol shown in FIG. 22.

FIG. 28 is a gel showing asymmetrically tagged E. coli genomic DNA fragments sorted for five cycles according to the SPE scheme shown in FIG. 22 (see Example I).

FIG. 29 provides data demonstrating that sorting according to the SPE scheme shown in FIG. 22 does not lead to biases in MID representation (as compared to the input MID-tagged polynucleotides). The ratio of sorted MID-tagged polynucleotides to input MID-tagged polynucleotides for each MID present in the pooled sample is shown after each of 5 sorting cycles. The four nucleotide sequence for each MID is shown on the X axis.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “TAQMAN™” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base pairing with a nucleotide in the other strand. A stable duplex can include Watson-Crick base pairing and/or non-Watson-Crick base pairing between the strands of the duplex (where base pairing means the forming hydrogen bonds). In certain embodiments, a non-Watson-Crick base pair includes a nucleoside analog, such as deoxyinosine, 2,6-diaminopurine, PNAs, LNA's and the like. In certain embodiments, a non-Watson-Crick base pair includes a “wobble base”, such as deoxyinosine, 8-oxo-dA, 8-oxo-dG and the like, where by “wobble base” is meant a nucleic acid base that can base pair with a first nucleotide base in a complementary nucleic acid strand but that, when employed as a template strand for nucleic acid synthesis, leads to the incorporation of a second, different nucleotide base into the synthesizing strand (wobble bases are described in further detail below). A “mismatch” in a duplex between two oligonucleotides or polynucleotides means that a pair of nucleotides in the duplex fails to undergo Watson-Crick bonding.

“Genetic locus,” “locus,” or “locus of interest” in reference to a genome or target polynucleotide, means a contiguous sub-region or segment of the genome or target polynucleotide. As used herein, genetic locus, locus, or locus of interest may refer to the position of a nucleotide, a gene or a portion of a gene in a genome, including mitochondrial DNA or other non-chromosomal DNA (e.g., bacterial plasmid), or it may refer to any contiguous portion of genomic sequence whether or not it is within, or associated with, a gene. A genetic locus, locus, or locus of interest can be from a single nucleotide to a segment of a few hundred or a few thousand nucleotides in length or more. In general, a locus of interest will have a reference sequence associated with it (see description of “reference sequence” below).

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whiteley et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Nucleoside” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structural Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-β-alkylribonucleotides, polynucleotides containing C-5 propynylpyrimidines, locked nucleic acids (“LNAs”), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature>90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“TaqMan”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified.

“Polynucleotide” or “oligonucleotide” is used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, wobble base pairing, or the like. As described in detail below, by “wobble base” is meant a nucleic acid base that can base pair with a first nucleotide base in a complementary nucleic acid strand but that, when employed as a template strand for nucleic acid synthesis, leads to the incorporation of a second, different nucleotide base into the synthesizing strand. Such monomers and their internucleosidic linkages may be naturally occurring or may be analogs thereof, e.g. naturally occurring or non-naturally occurring analogs. Non-naturally occurring analogs may include peptide nucleic acids (PNAs, e.g., as described in U.S. Pat. No. 5,539,082, incorporated herein by reference), locked nucleic acids (LNAs, e.g., as described in U.S. Pat. No. 6,670,461, incorporated herein by reference), phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for maximum efficiency in amplification, but may alternatively be double-stranded. If double-stranded, the primer is usually first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

“Readout” means a parameter, or parameters, which are measured and/or detected that can be converted to a number or value. In some contexts, readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

As used herein, the term “Tₘ” is used in reference to the “melting temperature.” The melting temperature is the temperature (as measured in ° C.) at which a population of double-stranded nucleic acid molecules becomes half dissociated into single strands. Several equations for calculating the Tm of nucleic acids are well known in the art. As indicated by standard references, a simple estimate of the Tm value in degrees centigrade may be calculated by the equation. Tₘ=81.5+0.41 (% (G+C)), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

“Sample” means a quantity of material from a biological, environmental, medical, or patient source in which detection, measurement, or labeling of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

By “terminator”, “terminating nucleotide”, “nucleic acid synthesis terminator” and variations thereof is meant a nucleotide that can be incorporated into a primer (or polymerizing nucleic acid strand) by a polymerase extension reaction, wherein the nucleotide prevents subsequent incorporation of nucleotides to the primer and thereby halts polymerase-mediated extension. Typical terminators are nucleoside triphosphates that lack a 3′-hydroxyl substituent and include 2′,3′-dideoxyribose, 2′,3′-didehydroribose, and 2′,3′-dideoxy-3′-haloribose, e.g. 3′-deoxy-3′-fluoro-ribose or 2′,3′-dideoxy-3′-fluororibose nucleosides, for example. Alternatively, a ribofuranose analog can be used in terminators, such as 2′,3′-dideoxy-β-D-ribofuranosyl, β-D-arabinofuranosyl, 3′-deoxy-β-arabinofuranosyl, 3′-amino-2′,3′-dideoxy-β-ribofaranosyl, and 2,3′-dideoxy-3′-fluoro-β-ribofuranosyl. A variety of terminators are disclosed in the following references: Chidgeavadze et al., Nucleic Acids Res., 12: 1671-1686 (1984); Chidgeavadze et al., FEBS Lett., 183: 275-278 (1985); Izuta et al, Nucleosides & Nucleotides, 15: 683-692 (1996); and Krayevsky et al, Nucleosides & Nucleotides, 7: 613-617 (1988). Nucleotide terminators also include reversible nucleotide terminators, e.g. Metzker et al. Nucleic Acids Res., 22(20):4259 (1994). Terminators may have a capture moiety, such as biotin, or a derivative thereof, e.g. Ju, U.S. Pat. No. 5,876,936, which is incorporated herein by reference. As used herein, a “predetermined terminator” is a terminator that basepairs with a pre-selected nucleotide of a template.

The terms “upstream” and “downstream” in describing nucleic acid molecule orientation and/or polymerization are used herein as understood by one of skill in the art. As such, “downstream” generally means proceeding in the 5′ to 3′ direction, i.e., the direction in which a nucleotide polymerase normally extends a sequence, and “upstream” generally means the converse. For example, a first primer that hybridizes “upstream” of a second primer on the same target nucleic acid molecule is located on the 5′ side of the second primer (and thus nucleic acid polymerization from the first primer proceeds towards the second primer).

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION OF THE INVENTION

The invention provides methods for amplifying and sorting polynucleotides based on predetermined sequence characteristics to form subpopulations of reduced complexity. In one aspect, such sorting methods are used to analyze populations of uniquely tagged polynucleotides, such as genome fragments. During or at the conclusion of repeated steps of amplification and sorting in accordance with the invention, the tags and the associated genomic sequences may be replicated, labeled and hybridized to a solid phase support, such as a microarray, to provide a simultaneous readout of sequence information from the polynucleotides. As described more fully below, predetermined sequence characteristics include, but are not limited to, a unique sequence region at a particular locus, a series of single nucleotide polymorphisms (SNPs) at a series of loci, or the like. In one aspect, such sorting of uniquely tagged polynucleotides allows massively parallel operations, such as simultaneously sequencing, genotyping, or haplotyping many thousands of genomic DNA fragments from different genomes.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It is noted that as used herein and in the appended claims, the singular forms “a”, “an”, and “the” include plural referents unless the context clearly dictates otherwise. Thus, for example, reference to “a nucleic acid” includes a plurality of such nucleic acids and reference to “the compound” includes reference to one or more compounds and equivalents thereof known to those skilled in the art, and so forth.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, A., Principles of Biochemistry 3^{rd} Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the present invention provides methods and compositions for amplifying and sorting polynucleotides based on predetermined sequence characteristics to form subpopulations of reduced complexity. Immortalized pooled polynucleotide samples and method of producing the same are also provided.

### Overview of Workflow

Below is a brief outline of the workflow for amplifying and sorting nucleic acids from a nucleic acid sample by selective primer extension, thereby producing one (or more) reduced complexity sample. While the subject invention finds use in the workflow described below, aspects of the invention find use in other applications.

The steps in an exemplary workflow include the following (some of which are described in further detail in following sections):

1. Asymmetrically tagging nucleic acid fragments to form a tagged sample. The asymmetric tags employed include functional domains (e.g., primer binding sites, polymerase binding sites, sequencing sites, amplification regions, etc.) and a Multiplex Identifier sequence (MID).

2. Amplifying/replicating the tagged nucleic acids.

3. Sorting the tagged nucleic acid fragments in the sample based on the presence (or absence) of a nucleotide or nucleotide sequence at a first position (consisting of one or two bases) in a sorting region.

4. Optionally re-amplifying the sorted fragments, e.g., to produce tagged fragments that can be further analyzed/sorted.

5. Optionally performing another sorting process based on the presence (or absence) of a nucleotide or nucleotide sequence at a second location (again consisting of one or two bases) in the same or different sorting region.

6. Analyzing the sorted fragments (e.g., subjecting one or more sorted fragment sample to sequencing, including “next generation” sequencing, e.g., Roche 454 sequencing).

In certain embodiments, the workflow includes enriching for one or more specific target nucleic acid sequence(s), e.g., specific adapter ligated fragment(s), at one or more steps in the workflow to enrich for one or more specific region of interest. In one embodiment, a previously sorted sample is further enriched for a sequence of interest (e.g., after performing steps 3 and 4 at least once). In another example, the parent population, either before or after adapter ligation, is enriched for fragments containing a region of interest (ROI). In most embodiments, ROI enrichment is performed after adapter ligation step 1. Any convenient method for producing a sample enriched for a nucleic acid having a region of interest may be employed.

For example, one or more species of nucleic acid fragment may be enriched (or isolated) from a sample by selective hybridization to one or more capture moieties (e.g., capture oligonucleotides complementary to a sequence in a ROI or capture antibodies, e.g., specific for a transcription factor; etc.). In such embodiments, the sample is contacted to the capture moiety (or moieties) to form target/capture moiety complexes (e.g., capture oligonucleotide hybridized to polynucleotides containing a nucleic acid sequence substantially complementary to the capture oligonucleotide). Unbound nucleic acid fragments are washed away from these complexes after which the captured target nucleic acid fragments are eluted. These eluted (enriched) nucleic acids can then be subjected to downstream processing (e.g., asymmetric tagging, amplification, sorting, etc.). Exemplary, non-limiting enrichment processes are described in U.S. Patent Application Publication 20060046251; U.S. Pat. No. 6,280,950; and PCT publication WO/2007/057652, all of which are incorporated by reference herein in their entirety.

In certain embodiments, the parent sample is processed into an “immortalized” sample in the workflow. In certain embodiments, an “immortalized” sample is a sample from which copies can be made without degrading the integrity of the original sample, akin to producing a “master copy” of a document from which photocopies can be made indefinitely. For example, an immortalized sample can be produced by immobilizing adapter-ligated fragments to a solid substrate, where the adapter includes a synthesis primer binding site. Such immobilized fragments can be used to produce copies of the fragments by primer extension using the adapter primer binding site. These copies can be eluted from the immobilized fragments for downstream manipulation. The immobilized adapter ligated fragments can then be used to produce more copies.

In certain embodiments, an immortalized sample is a sample that allows an indefinite number of sequential copies to be produced. This is akin to making copies from previous copies, for example as in producing a copy of a copy of an original electronic file. For example, a sample of nucleic acid fragments that include PCR primer binding sites on both ends (e.g., present in adapter sequences ligated to the fragments) can be PCR amplified to produce a first copy of the fragments, the first copy can be PCR amplified to produce a second copy, etc. As described in detail below, other functional sites in one or both adapter sequences on the terminal ends of nucleic acid fragments can be used to form immortalized samples. For example, one adapter sequence may include a copying initiation site, such as nucleic acid synthesis primer binding site (e.g., for linear amplification using phi29 DNA polymerase) or a promoter sequence (e.g., T7 or T3 polymerase binding site). As another example, the first adapter and second adapter each may include a copying initiation site (e.g., opposing T3 and T7 polymerase binding sites, nucleic acid synthesis primer binding sites, or combinations thereof). The specific functional site(s), placement and orientation in the adapter sequences is up to the desires of the user.

In certain embodiments, an adapter ligated sample includes functional elements that allow it to be immortalized both of the ways described above (i.e., such that the original adapter ligated sample can be copied indefinitely and such that the resultant copies can be copied sequentially, e.g., as is done when performing multiple rounds of sorting by Selective Primer Extension (SPE) described in detail below).

### Asymmetrically Tagging Nucleic Acid Fragments

### Sources of the Nucleic Acids to be Processed

Nucleic acids in a nucleic acid sample being analyzed (or processed) in accordance with the present invention can be from any nucleic acid source. As such, nucleic acids in a nucleic acid sample can be from virtually any nucleic acid source, including but not limited to genomic DNA, complementary DNA (cDNA), RNA (e.g., messenger RNA, ribosomal RNA, short interfering RNA, microRNA, etc.), plasmid DNA, mitochondrial DNA, etc. Furthermore, as any organism can be used as a source of nucleic acids to be processed in accordance with the present invention, no limitation in that regard is intended. Exemplary organisms include, but are not limited to, plants, animals (e.g., reptiles, mammals, insects, worms, fish, etc.), bacteria, fungi (e.g., yeast), viruses, etc. In certain embodiments, the nucleic acids in the nucleic acid sample are derived from a mammal, where in certain embodiments the mammal is a human.

In certain embodiments, the nucleic acid sample employed is an enriched sample. By enriched sample is meant that the nucleic acid sample has been subjected to a process that selects for nucleic acids having a particular feature. Generally, an enriched sample has an increase in the relative concentration of particular nucleic acid species in the sample based on, e.g., having a specific region of interest, including a specific nucleic acid sequence, lacking a specific locus or sequence, being within a specific size range, etc. There are a wide variety of ways to enrich nucleic acids having a specific characteristic(s) or sequence, and as such any convenient method to accomplish this may be employed.

In certain embodiments, nucleic acids in the nucleic acid sample are amplified prior to analysis. In certain of these embodiments, the amplification reaction also serves to enrich a starting nucleic acid sample for the locus of interest. For example, a starting nucleic acid sample can be subjected to a polymerase chain reaction (PCR) that amplifies one or more region of interest. In certain embodiments, the amplification reaction is an exponential amplification reaction whereas in certain other embodiments, the amplification reaction is a linear amplification reaction. Any convenient method for performing amplification reactions on a starting nucleic acid sample can be used in practicing the subject invention. In certain embodiments, the nucleic acid polymerase employed in the amplification reaction is a polymerase that has proofreading capability (e.g., phi29 DNA Polymerase, Thermococcus litoralis DNA polymerase, Pyrococcus furiosus DNA polymerase, etc.).

In certain embodiments, the nucleic acid sample being analyzed is derived from a single source (e.g., a single organism, tissue, cell, subject, etc.), whereas in other embodiments, the nucleic acid sample is a pool of nucleic acids extracted from a plurality of sources (e.g., a pool of nucleic acids from a plurality of organisms, tissues, cells, subjects, etc.), where by “plurality” is meant two or more. As such, in certain embodiments, a nucleic acid sample can contain nucleic acids from 2 or more sources, 3 or more sources, 5 or more sources, 10 or more sources, 50 or more sources, 100 or more sources, 500 or more sources, 1000 or more sources, 5000 or more sources, up to and including about 10,000 or more sources. As described above, the nucleic acids in nucleic acid samples from a single source as well as from multiple sources include a locus of interest for which at least one reference sequence is known.

In certain embodiments, nucleic acid fragments tagged according to aspects of the subject invention are to be pooled with nucleic acid fragments derived from a plurality of sources (e.g., a plurality of organisms, tissues, cells, subjects, etc.), where by “plurality” is meant two or more. In such embodiments, the asymmetric adapters employed for each separate nucleic acid sample may include a uniquely identifying sequence (or Multiplex Identifier; MID) such that after the tagging process is complete, the source from which the each tagged nucleic acid fragment was derived can be determined. Any type of uniquely identifying sequence/MID can be used, including but not limited to those described in co-pending U.S. patent application Ser. No. 11/656,746, filed on Jan. 22, 2007, and titled “Nucleic Acid Analysis Using Sequence Tokens”, as well as U.S. Pat. No. 7,393,665, issued on Jul. 1, 2008, and titled “Methods and Compositions for Tagging and Identifying Polynucleotides”, both of which are incorporated herein by reference in their entirety for their description of specific nucleic acid sequences and their use in identifying polynucleotides. In certain embodiments, the identification sequences employed need not have any particular common property (e.g., Tₘ, length, base composition, etc.), as the asymmetric tagging methods (and many sequence readout methods, including but not limited to actual sequencing of the identifying DNA sequence or measuring the length of the DNA sequence identifier) can accommodate wide variety of unique identifying sets.

### Asymmetric Adapters

An “asymmetric adapter” as used herein is an adapter that when ligated to both ends of a double stranded nucleic acid fragment will lead to the production of amplification or copying products of the fragment that have non-identical adapter sequences. Thus, replication of an asymmetric adapter attached fragment(s) results in polynucleotide products with first and second adapters (on opposing ends of the fragment) having at least one nucleic acid sequence difference. In other words, the adapter on one end of a nucleic acid fragment produced according to methods of the present invention has at least one region or domain that has a nucleic acid sequence that is different from the adapter sequence on the other end.

Any convenient asymmetric adapter may be employed in practicing the present invention. Exemplary asymmetric adapters are described in: U.S. Pat. Nos. 5,712,126 and 6,372,434; U.S. Patent Publications 2007/0128624 and 2007/0172839; and PCT publication WO/2009/032167; all of which are incorporated by reference herein in their entirety. In certain embodiments, the asymmetric adapters employed are those described in U.S. patent application Ser. No. 12/432,080, filed on Apr. 29, 2009, incorporated herein by reference in its entirety.

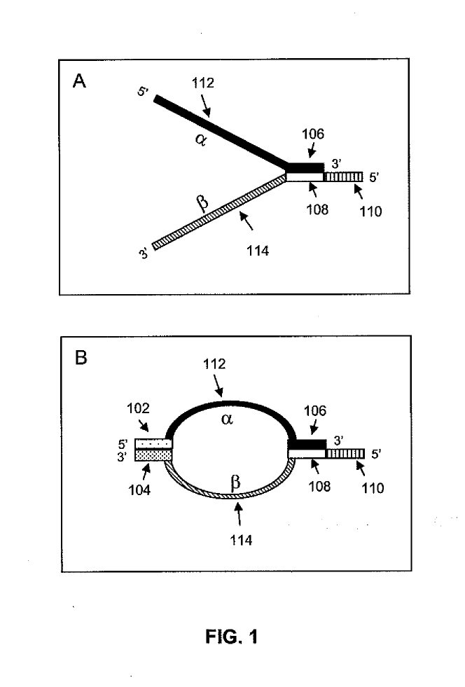

In certain embodiments, asymmetric adapters that find use in the present invention include one or more clamp regions, a ligation site adjacent to one of the clamp regions, and a region of substantial non-complementarity. FIG. 1 shows two embodiments for asymmetric adapter structures that are described in U.S. patent application Ser. No. 12/432,080). The asymmetric adapter in FIG. 1A includes two nucleic acid strands: a top strand having elements 112 and 106 in a 5′ to 3′ orientation, and a bottom strand having elements 114, 108 and 110 in a 3′ to 5′ orientation. As is evident from the structure shown in FIG. 1A, elements 106 and 108 hybridize to one another forming a first clamp region that, when ligated to a compatible end of a nucleic acid fragment via ligation site 110 (discussed below), is proximal to the nucleic acid fragment (also referred to as “inner”). As such, the sequence of element 106 is complementary to the sequence of element 108. The asymmetric adapter in FIG. 1B also includes two nucleic acid strands: a top strand having elements 102, 112, and 106 in a 5′ to 3′ orientation, and a bottom strand having elements 104, 114, 108, and 110 in a 3′ to 5′ orientation. As with the structure shown in FIG. 1A, elements 106 and 108 in FIG. 1B hybridize to one another forming a first clamp region that is proximal to the nucleic acid fragment once ligated thereto (also referred to as “inner”). Unlike the asymmetric adapter in FIG. 1A, the asymmetric adapter in FIG. 1B includes elements 102 and 104 which hybridize to one another forming a second clamp region that is distal to the nucleic acid fragment (also referred to as “outer”). As such, the sequence of element 102 is complementary to the sequence of element 104 and the sequence of element 106 is complementary to the sequence of element 108. The length of such complementary regions which form clamp structures in the asymmetric adapters can vary and, in certain embodiments, can be affected by other sequences in the vectorette adapter, e.g., the region of substantial non-complementarity (also referred to as the “asymmetric” region). In certain embodiments the length of the complementary sequence is from 6 nucleotides to 50 nucleotides. For example, predictions based on a 2-state hybridization model indicate that 6 bases of complementarity (having the sequence 5′ CTCCTC 3′ on the top strand) would be sufficient to form a proximal clamp region under the following conditions: 50 mM NaCl, 10 mM MgCl₂, 10 uM adapter at 20° C.

The asymmetric adapter structures in FIGS. 1A and 1B include one or more region of substantial non-complementarity represented by elements 112 and 114 (denoted as regions α and β, respectively). This region is also referred to herein as an “asymmetric” region. By substantially non-complementary is meant that one or both of elements 112 and 114 include at least one region of nucleic acid sequence that is not complementary to the other strand. The length and identity of the one or more region of non-complementarity will vary based on the desires of the user (e.g., based on the downstream analyses to be performed on the resultant asymmetrically tagged nucleic acid). For example, in certain embodiments, elements 112 and 114 (or α and β) include one or more particular sequences which are useful for later steps in the workflow. Such sequences include, but are not limited to, restriction enzyme sites, PCR primer binding sites, linear amplification primer sites, NuGEN SPIA® primer sites, reverse transcription primer sites, RNA polymerase promoter sites (such as for T7, T3 or SP6 RNA polymerase), unique identity sequences (e.g., sequences employed to mark the nucleic acid fragment as being derived from a specific starting sample), sequencing primer sites, reflex sequences, etc. Reflex sequences find use in performing intramolecular rearrangement to place a region of interest in proximity to a functional domain (e.g., a domain in an adapter, e.g., a primer binding site or MID). The reflex process is described in detail in U.S. provisional applications 61/235,595, and 61/288,792, filed on Aug. 20, 2009 and Dec. 21, 2009, respectively, and entitled “Compositions and Methods for Intramolecular Nucleic Acid Rearrangement Using Reflex Sequences”, incorporated herein by reference.

In certain embodiments, the MID in an asymmetric adapter is a DNA sequence which uniquely identifies the sample or sample region from which the fragment so labeled originates. It is noted here that there are no constraints with regard to members of a set of MIDs being employed in the present invention. For example, a set of MIDs that finds use in the subject invention need not have similar thermodynamic or physical properties between them, e.g., be isothermal.

As indicated above, the asymmetric adapters include a ligation site 110 that is adjacent to the first, proximal clamp region (formed by 106 and 108). The ligation site comprises a region of single-strandedness that selectively associates with a compatible end of the nucleic acid fragments. The compatible region of single-strandedness may be on the bottom strand, forming a 5′ overhang (as shown in FIG. 1) or, in certain embodiments, be present on the top strand, forming a 3′ overhang. In order to promote ligation of the asymmetric adapter to a compatible nucleic acid fragment, the 5′ end of the ligation site is phosphorylated (not shown in FIG. 1). Therefore, as described above and shown in FIG. 1, the ligation site is configured to allow ligation of an asymmetric adapter to a compatible end of a nucleic acid fragment which is to be asymmetrically tagged.

Producing compatible ends on a nucleic acid fragment that is to be tagged with an adapter is sometimes referred to herein as “polishing”. Polishing the ends of nucleic acid fragments may include, but is not limited to, cutting with a restriction enzyme, shearing the nucleic acid, adding one or more nucleotides, removing one or more nucleotides, and adding or removing a phosphate group. The resultant compatible ends can have blunt or protruding/recessed ends (i.e., having compatible overhang regions), both terms being well known in the art.

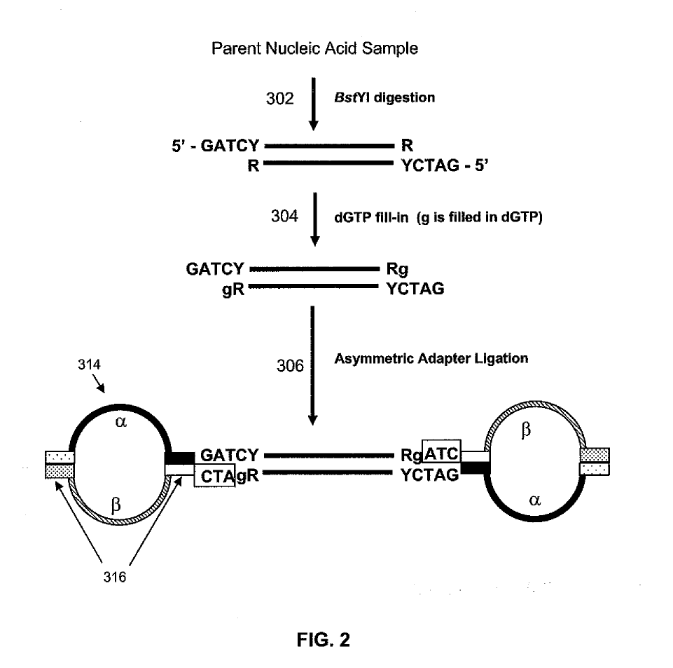

In certain embodiments, compatible ends of a nucleic acid fragment are produced by contacting a parent nucleic acid sample with a restriction enzyme and polishing the ends to make them compatible with the asymmetric adapter being employed (e.g., by adding a single base). As such, in these embodiments, the restriction enzyme generates nucleic acid fragments having cut sites on the ends that are compatible to the single stranded region of the asymmetric adapter, i.e., the ends of the nucleic acid fragments have regions of complementarity to the region of single-strandedness (i.e., the overhang regions at the cut site) in the ligation site of the asymmetric adapter. In this way, the asymmetric adapter ligation site and compatible ends of the nucleic acid fragments can be ligated to one another under appropriate ligation conditions (e.g., in the presence of an enzyme having DNA ligase activity in appropriate buffering conditions and co-factors). See, e.g., FIG. 2, described in detail below.

In certain embodiments, compatible ends of the nucleic acid fragments are not produced by restriction enzyme digestion. For example, a parental nucleic acid sample can be fragmented by applying shear forces to the sample, which leads to fragmented DNA. Polishing of the ends of such fragmented DNA can then be performed to produce blunt ends having no 5′ or 3′ overhang (e.g., by filling in and or removing overhangs as is known in the art). Asymmetric adapters compatible with such blunt-end fragments will themselves be blunt ended at the ligation site and have a 5′ phosphate group. In these embodiments, the blunt ends of the fragmented nucleic acid are de-phosphorylated to prevent fragment concatenation.

In certain other embodiments, a blunt end nucleic acid fragment(s), whether produced by shearing or by a restriction enzyme the produced blunt ends, is contacted with a DNA polymerase that can add a single specific nucleotide in a non-template dependent manner (e.g., an added dA to the 3′ end of blunt fragment using Taq polymerase). The compatible asymmetric adapter in such embodiments will be designed to have a compatible end containing a single base overhang that is complementary to the nucleotide added to the blunt ends of the fragment (e.g., the asymmetric adapter ligation site will have a 3′ dT overhang). This embodiment is akin to TA cloning systems employed for cloning Taq polymerase produced PCR products.

As is clear from the description above, any convenient method for creating compatible ends between nucleic acid fragments and asymmetric adapters to promote ligation of the asymmetric adapter while reducing inter-fragment ligation may be used.

### Basic Asymmetric Tagging Method

FIG. 2 shows steps in an exemplary method for asymmetrically tagging a nucleic acid fragment using asymmetric adapters as described above. It is noted here that other methods for asymmetrically tagging nucleic acid fragments have been described that do not employ the specific asymmetric adapters described above. For example, U.S. Pat. No. 7,217,522 and U.S. Patent Publication No. 2009/0004665 (incorporated by reference herein in their entirety) describe methods that employ methylase treatment and methylase-sensitive and methylase resistant restriction enzymes to asymmetrically tag nucleic acid fragments. Additional methods and compositions for producing fragments labeled with asymmetrical adapters can be found in U.S. Pat. Nos. 7,217,522; 7,365,179; and 7,393,665; and Meyer, et al. From micrograms to picograms: quantitative PCR reduces the material demands of high-throughput sequencing, Nucl. Acids Res. 2008 v36: e5 (doi:10.1093/nar/gkm1095; Advance Access published on Dec. 15, 2007); all of which are incorporated herein by reference in their entirety.

In the example shown in FIG. 2, a parent nucleic acid sample containing starting nucleic acid (e.g., genomic DNA) is digested in step 302 with a restriction enzyme (in this case BstYI) producing 5′ GATC overhang (BstYI has a recognition site of R/GATCY, where R is a purine and Y is a pyrimidine as conventionally denoted in the art and the slash indicating the position of the cut site). (It is noted here that the restriction enzyme Sau3AI also produces a 5′ GATC overhang).

At step 304, the 5′ GATC overhang is filled in with dG on the bottom strand (shown as “g”), producing a 5′ GAT overhang. This overhang represents the compatible end of the nucleic acid fragment that will serve as a ligation site for a suitably designed asymmetric adapter (i.e., one having a 5′ ATC overhang). The fill-in step 304 prevents the restriction-digested, double-stranded fragments of the starting nucleic acid sample from being ligated to each other during the asymmetric adapter ligation step (i.e., prevents inter-fragment ligation) as well as providing a 5′ phosphate which promotes sealing both top and bottom nicks.

It is noted here that there are numerous ways in which to produce nucleic acid fragments having ends compatible with an asymmetric adapter (or “polishing”, as described above). Polishing can include any number of manipulations, e.g., cutting with a restriction enzyme, shearing the nucleic acid, adding one or more nucleotides, removing one or more nucleotides, and adding or removing a phosphate group. The resultant compatible ends can have blunt or sticky ends (i.e., having compatible overhang regions), both terms being well known in the art.

In certain embodiments, a nucleic acid fragment may be ligated to two independent and distinct asymmetric adapters, each of which is ligated to a different compatible end of a nucleic acid fragment. Any convenient method for producing a nucleic acid fragment(s) having more than one distinct compatible end can be employed. In certain of these embodiments, the different compatible ends of the nucleic acid fragment are produced by digesting the nucleic acid fragment with more than one restriction enzyme. These multiply-digested fragments are ligated to separate asymmetric adapters, each of which will ligate to one of the compatible ends. The ligation of the asymmetric adapters can be sequential or simultaneous. In addition, more than two asymmetric adapters may be used to tag a nucleic acid sample containing multiple fragments with any variety of different compatible ends. This will depend on the desires of the user and the specific analyses to be performed on the resultant asymmetrically tagged nucleic acid fragments.

In step 306, asymmetric adapter 314 having 5′ATC overhang (shown in the box) is ligated to the nucleic acid fragments having compatible 5′GAT overhangs on both ends. The asymmetric adapters shown include two clamp regions 316 (proximal and distal, with respect to their position relative to the nucleic acid fragment once ligated to it) formed by compatible ends of the two strands of the asymmetric adapter. The top strand of the asymmetric adapter includes a region of substantial non-complementarity designated as a and the bottom strand of the asymmetric includes a region of substantial non-complementarity designated as β. In other words, α and β are not fully complementary sequences, and as such do not form a continuous hybridized structure. As described above, regions α and β may include specific regions that facilitate or allow specific downstream analyses as desired by a user of the method.

### DNA-Based Amplification and Sorting of Asymmetrically Tagged Nucleic Acids

Once asymmetrically tagged nucleic acids are produced, they may be employed in any variety of different assays, where the specific downstream steps will vary depending on the desired outcome of the assay.

In certain embodiments, after adapter ligation, the tagged nucleic acids are amplified (or replicated). It is noted here that if a plurality of different nucleic acid samples have been tagged with asymmetric adapters, each sample comprising an adapter with a different MID corresponding to the sample, all or part of each sample may be combined into a single pooled sample prior to the first amplification step (described below). In certain other embodiments, each different sample may be amplified prior to combining. Regardless of when the samples are combined (if combining different tagged samples is desired), a variety of methods for performing the amplification step can be used. Non-limiting examples of certain amplification methods are described in further detail below.

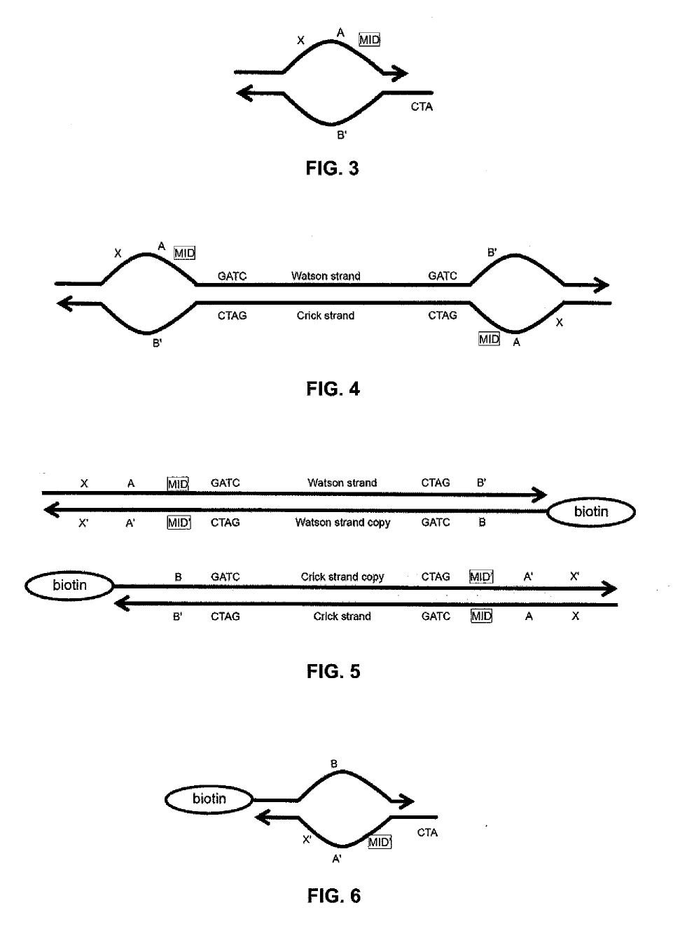

In the following description of exemplary amplification reactions, the asymmetric adapter will be represented as shown in FIG. 3. In FIG. 3, the bottom strand of the asymmetric adapter has a 5′ phosphorylated ATC overhang (indicated by CTA on the right side of the bottom strand), which allows it to anneal and become ligated to the filled-in fragments as previously described. The arrows on the upper and lower strands of the asymmetric adapter in FIG. 3 (and following Figures) denote the 5′ to 3′ direction of each strand (the direction of nucleic acid synthesis). The non-complementary region in the asymmetric adapter is shown as a “bubble” region in FIG. 3 and includes certain functional elements. In the asymmetric adapter of FIG. 3, these elements include: 1) primer binding site X, which finds use in, for example, linear amplification of the fragments (as described below); 2) Roche 454 Sequencing System A and B sequencing primer binding sites, where the B site in the lower strand is denoted B′ to convey that it is complementary to the Roche 454 B sequencing site (it is noted that any number of different sequencing primer binding sites may be employed, e.g., for the Illumina platform); and 3) a MID, as described above. It is noted here that these elements are merely exemplary, and that in other embodiments, certain elements may be eliminated or added as desired by the user.

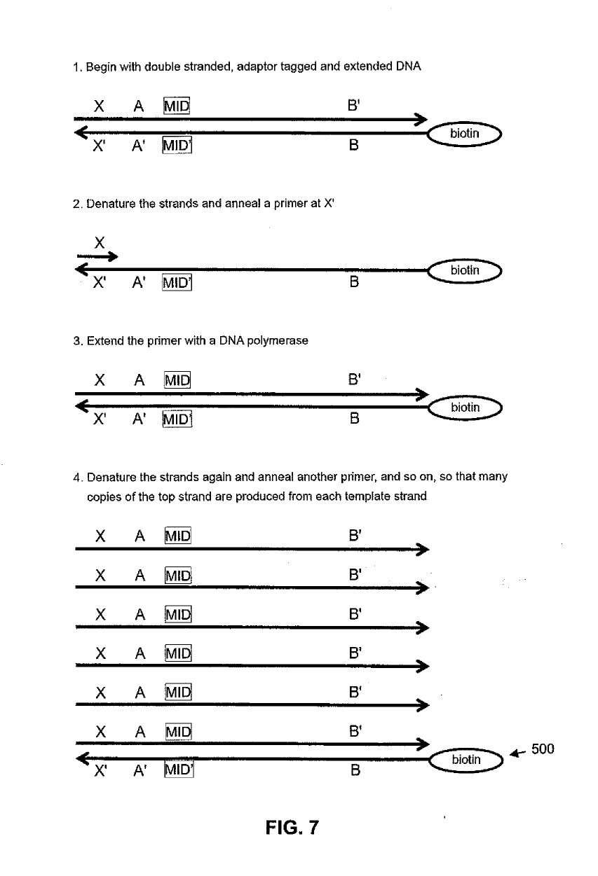

FIG. 4 shows the structure of a tagged, double-stranded nucleic acid fragment produced by restriction enzyme digestion followed by ligation of the asymmetric adapter shown in FIG. 3 to both ends. The upper and lower strands of the nucleic acid fragment are denoted “Watson strand” and “Crick strand” to keep track of the fragment strands in later steps. As noted above, the digested and tagged nucleic acid fragments may be pooled with other such tagged DNAs at this point.

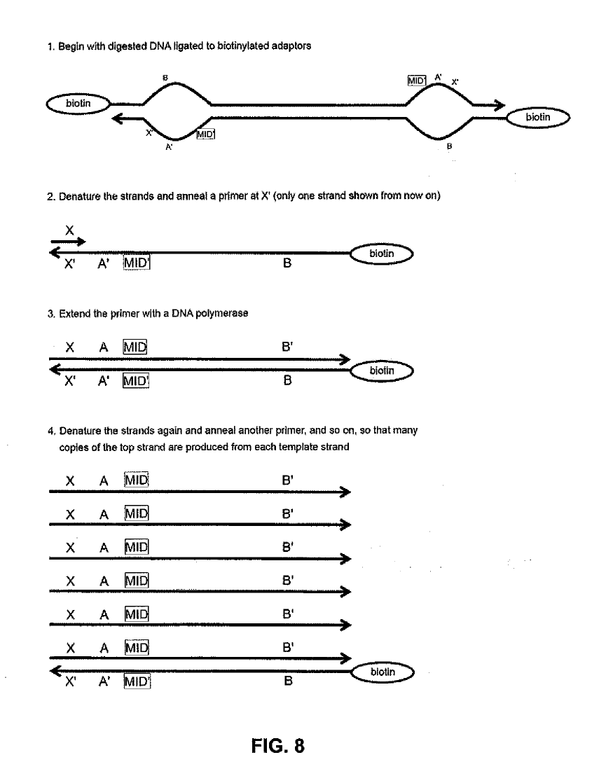

The next step in the current workflow is performing a primer extension reaction with a primer annealing at the 3′ end of B′. The primer may prime completely within B′ (starting at the 3′ boundary of B′), partially within in B′ (part upstream of B′ and part within B′) or completely upstream of B′. This extension reaction results in fully double stranded DNA fragments that are asymmetrical with respect to the orientation of their adapters. In certain embodiments (and as shown in FIG. 5), the primer employed in this step is modified with a biotin at its 3′ end (also referred to as a binding moiety, or a member of a binding pair) which facilitates the removal of the extended strand before the sorting reaction (e.g., using an avidin-bound substrate). FIG. 5 shows both orientations of the resulting biotinylated extension products. In certain embodiments, the primer is conjugated to binding moieties other than biotin.

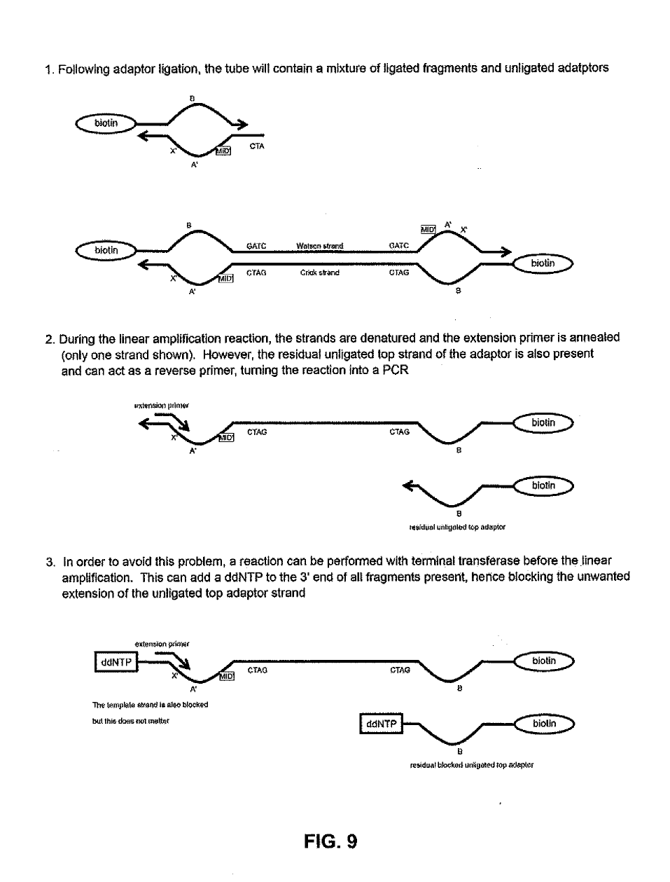

However, it is also possible to produce a biotinylated template for amplification by putting a biotin modification directly onto the adapter. In this case, the adapter structure is reversed as shown in FIG. 6. The biotinylated adapters can be annealed and ligated to the digested and filled-in DNA fragments as before, and these can be used directly in linear amplification reactions as outlined below (FIG. 8).

Thermal Cycling-Based Amplification

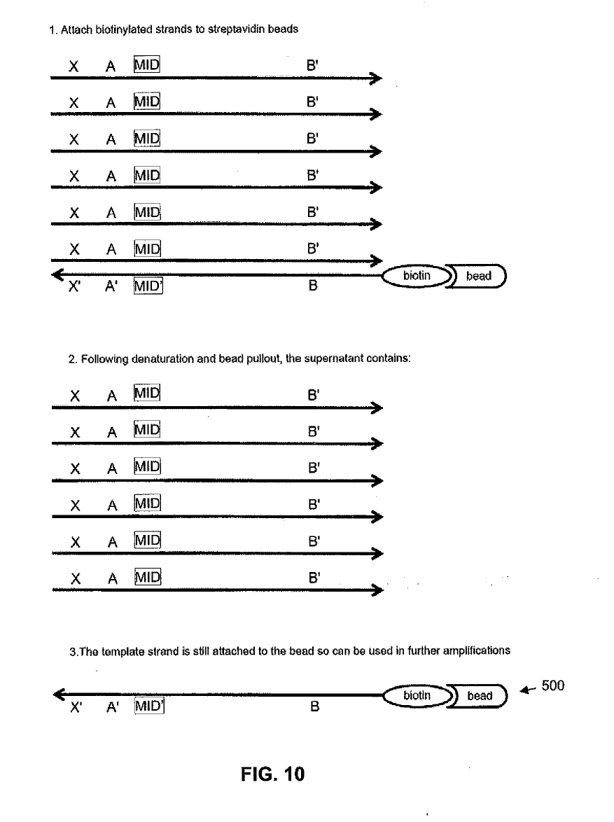

FIG. 7 shows an exemplary thermal-cycling based linear amplification of asymmetrically tagged nucleic acids. For purposes of clarity, only amplification of the Watson strand and its copy from FIG. 5 is shown. In addition, certain specific sequence elements of the asymmetrically tagged nucleic acid have been excluded.

In step 1, the double stranded template is heat-denatured; in step 2, the temperature is reduced to allow annealing synthesis primer X to the template strand at its complementary site X′; at step 3, the annealed X primer is extended by a thermostable DNA polymerase (e.g., after raising the temperature to an optimal level for DNA synthesis); at step 4, the denaturation, annealing and extension reactions are repeated until a desired number of rounds have been completed. Because there is only one synthesis primer in the reaction (i.e., primer X), the steps shown in FIG. 7 result in a linear, rather than exponential, amplification of the template strand.

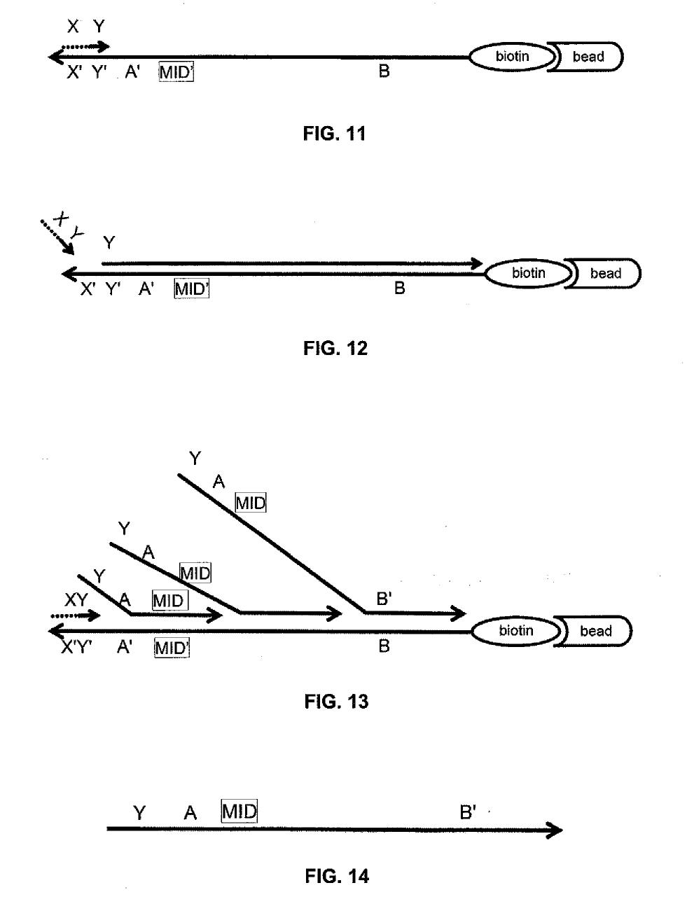

In certain embodiments (and as indicated above), when employing the biotinylated adapters shown in FIG. 6, linear amplification may be performed directly from the adapter ligated fragments. When using the adapter ligated fragments directly, the mixture will also contain excess adapter that has failed to ligate onto the digested and filled-in end of the DNA fragments. This may confound the subsequent linear amplification reaction as the top strand of the adapter can act as a reverse primer and turn the linear amplification reaction into an exponential PCR (i.e., the excess top strand of the adapter and the linear amplification primer form a PCR primer pair for the adapter-ligated fragment template; see FIG. 9, step 2). The top strand may also act as a forward primer because of the inner stem region. In order to overcome this problem, an additional step using the enzyme terminal transferase can be performed prior to the linear amplification step (i.e., prior to adding the amplification primer). Specifically, terminal transferase can be employed to add a di-deoxynucleotide to the 3′ end of DNA in the adapter ligated sample in a template independent manner. Following such a reaction, the unligated top strand of the adapter will be unable to participate in the linear amplification (see FIG. 9, step 3). Purification of the substrate library to remove free adapter can also be employed to achieve the same goal.

In certain embodiments, non-biotinylated adapter ligated nucleic acid fragments can be used directly in linear amplification reactions using a non-biotinylated synthesis primer. The non-biotinylated nucleic acids produced in this direct linear amplification can be sorted according to aspects of the subject invention using specially modified sorting primers (as detailed below).

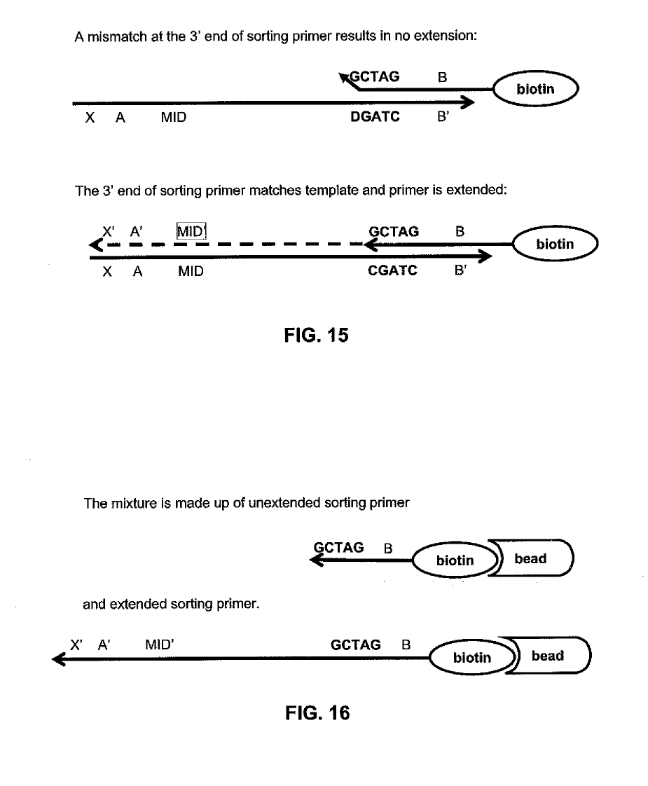

In certain embodiments, the template strand is removed before performing any subsequent assay steps using on the amplified sample. In embodiments in which a binding moiety is attached to the extension primer used in the extension reaction (as shown in FIG. 7), template strand removal is achieved by binding the biotinylated strands to streptavidin beads (FIG. 10). Treatment with denaturing conditions such as solutions of sodium hydroxide, high temperature, etc. denatures any double stranded material and the copied strands can be eluted into the supernatant, leaving the biotinylated template strands on the beads (note that this denaturation step may be omitted as it is likely that there will be sufficient single stranded copies already present in the solution phase). These template strands may be viewed as the ‘immortalized’ fragment library and can be used as templates for further amplifications if desired.

The eluted material is suitable for sorting (discussed further below) or alternatively can be used directly for region of interest (ROI) extraction (or enrichment) and sequencing. Note also that linear amplification could be used following ROI extraction in order to increase the amount of material available for next generation sequencing or other sequence analysis approaches including Sanger sequencing and use of nucleic acid microarrays.

NuGEN-Based Amplification

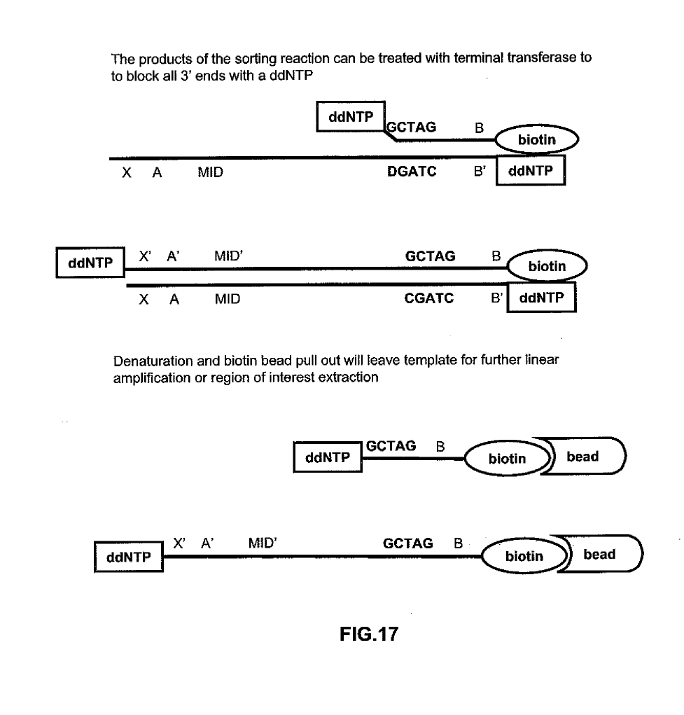

The linear amplification discussed in this section relies upon the use of NuGEN's chimeric RNA-DNA primers (e.g., as described in U.S. Pat. Nos. 6,251,639 and 6,692,918, incorporated herein by reference in their entirety). The RNA portion X is located at the 5′-end of the oligonucleotide (indicated by the dotted line in FIG. 11) and anneals to X′ while the DNA portion Y is located at the 3′-end and anneals to Y′ (for purposes of clarity, not all sequence labels are included in these figures).

A strand displacing DNA polymerase extends the annealed primer. At the same time, RNase H digests the RNA portion of the extended primer, revealing an annealing site for another SPIA® primer (FIG. 12). The new primer is extended and the strand displacement activity of the polymerase pushes the previous copy off the template. Again, RNase H digests the RNA portion of the newly extended primer, thereby allowing another primer to anneal and extend, and so on (FIG. 13).

Denaturation of the strands followed by a bead pullout leaves the copied material in the supernatant and the template strands are available for further use if necessary. The resulting amplification products (FIG. 14) contain the Y sequence but have lost the X sequence, which is required if these fragments are to be used in further rounds of amplification. In certain embodiments, the X sequence is reconstituted, with exemplary embodiments for doing so described in detail below). This material is suitable for sorting (as described below) or alternatively can be used directly for region of interest (ROI) extraction and sequencing. Note also that SPIA® could be used following ROI extraction in order to increase the amount of material available for next generation sequencing or other sequence analysis approaches including Sanger sequencing and use of nucleic acid microarrays.

Sorting DNA-based Amplification Samples Using Selective Primer Extension

The purpose of the sorting reaction is to systematically reduce the complexity of the fragment library in order to make it more amenable to subsequent processing steps or reactions, e.g., culling and/or region of interest (ROI) extraction. As described above, ROI extraction is a process by which polynucleotides in a sample that include a specific region or locus (e.g., a gene of interest) are isolated, e.g., using a capture oligonucleotide complementary to a sequence within the ROI. By “culling” is meant a process by which polynucleotides possessing a specific nucleotide or nucleotide sequence at a locus are isolated (e.g., polynucleotides having a specific nucleotide/sequence at a sorting region or an SNP, mutation or other variation at a specific genomic locus/loci; see, e.g., U.S. provisional Patent Application 61/258,143 filed Nov. 4, 2009 and titled “Base by Base Mutation Screening”).

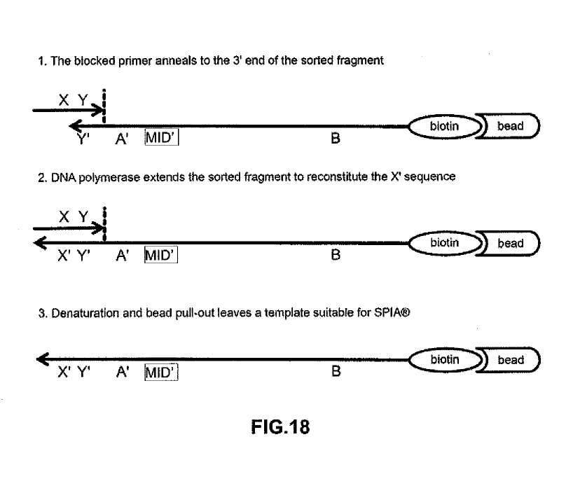

One exemplary method for performing a sorting reaction is shown in FIG. 15. In this example, the known adapter sequences are exploited to allow a sorting primer to interrogate the identity of the nucleotide adjacent to the restriction enzyme cut site used to generate the asymmetrically tagged polynucleotides and sort accordingly. As shown in FIG. 15, the bases that follow the GATC cut site on the ‘B’ side of the fragment are the ones used to sort the polynucleotides (it is noted that sequences other than GATC will be present in adapter-ligated polynucleotides when using restriction enzymes that cut at different sites; as such no limitation in this regard is intended). In the first round, fragments containing C in the first sorting position of the template strand can be sorted from those containing D (where D refers to any base except C). From this ‘C’ pool, following a further round of amplification, fragments containing CA can be sorted using a sorting primer indexed for the second sorting position (i.e., having bases complementary to CA at the 3′ end), and so on. In certain embodiments, the system involves five rounds of sorting using indexed sorting primers, i.e., sorting up to position NNNNN following the GATC site.

The basic sorting process shown in FIG. 15 (which is exemplary and not meant to be limiting) is driven by a process termed selective primer extension (SPE). The SPE process exploits the inability of DNA polymerases to initiate synthesis from a primer having a mismatched terminal 3′ nucleotide (see, e.g., Low, et al. Analysis of the amplification refractory mutation allele-specific polymerase chain reaction system for sensitive and specific detection of p53 mutations in DNA, J Pathol 2000, 190:512-5; Hodgson, et al. ARMS™—Allele-specific Amplification based Detection of Mutant p53 DNA and mDNA in tumours of the breast. Clinical Chemistry 47(4):774-778; both of which are incorporated by reference herein in their entirety). In the SPE process, the fragments undergo an extension reaction with a primer annealing at B′, which in the first round extends one base beyond the GATC site on that side of the fragment. In certain embodiments, the primer (also called a sorting primer) is protected via a phosphorothioate (PTO) or locked nucleic acid (LNA) modification at its 3′ end in order to prevent digestion by the polymerase enzyme, which in some embodiments possesses 3′ to 5′ exonuclease activity (e.g., Vent DNA polymerase; this characteristic leads to improved accuracy of the enzyme). Note that the PTO modification produces oligonucleotides in two isomers, only one of which is protected from digestion. To ensure that all oligonucleotides used are of the protected form, a pre-digestion reaction is performed on the PTO-modified oligos with an exonuclease enzyme to remove the unprotected isomer. The combination of a proofreading polymerase, a 3′ protected primer, and a suitable annealing temperature means that the primer will only be extended when it is annealed to a fragment containing the base complementary to its terminal 3′ base. For example, if the sorting primer has a G at its 3′ end, then only fragments containing a C at the corresponding location will be extended (FIG. 15).

It is also possible to sort by two bases simultaneously by using a sorting primer extending two bases into the sorting region. For example, to sort for fragments containing 5′-AG-3′ in the sorting region, a primer ending 5′-GATCCT-3′ would be used. This has the advantage that fewer cycles of sorting and amplification are required to sort all possible nucleotide sequences at the sorting site, but it also requires a higher fold of amplification as the fragments will be sorted into 16 pools instead of four (i.e., one pool for each possible two nucleotide combination of nucleotides A, G, C and T, where all combinations are sorted for in a cycle). The sorting reaction products are then bound to streptavidin beads, the strands are denatured and the attached biotinylated fragments are pulled out of the solution on the beads (FIG. 16). The attached extended material is suitable for input into another round of linear amplification, which in turn could be followed either by another sort or by ROI extraction and sequencing. If being used in another round of linear amplification, the material would enter the cycle in step 2 of FIG. 7 and the amplification would be performed with the template still attached to the streptavidin bead.

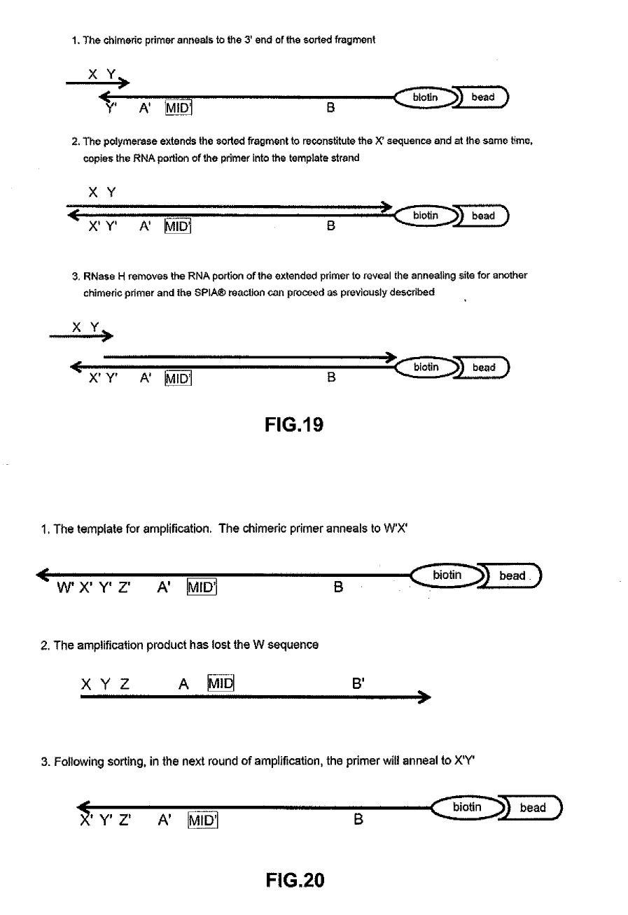

The extended material is suitable for input into another round of linear amplification, which in turn could be followed either by another sort or by ROI extraction and sequencing. However, before this can occur, the unextended sorting primer should be blocked to prevent it acting in the following linear amplifications or region of interest extractions as a reverse PCR primer. This is undesirable as, although providing a greater degree of amplification than the linear reactions described, PCR also exhibits bias towards shorter fragments. To avoid this problem, an additional reaction using the enzyme terminal transferase can be performed to add a blocking di-deoxynucleotide residue to the 3′-end of any DNA present (FIG. 17). Residual ddNTPs could then be removed by digesting with an enzyme such as shrimp alkaline phosphatase (SAP) in order to prevent them participating in subsequent reactions. If being used in another round of linear amplification, the material would enter the cycle in step 2 of FIG. 7 and the amplification could be performed with the template still attached to the streptavidin bead or released from it.

As indicated above, the fragments to sort can be produced directly from non-biotinylated adapter-ligated fragments using a non-biotinylated synthesis primer. In these embodiments, the sorting primer employed in SPE may be modified.

In one embodiment of this method, the sorting primer may be conjugated to a binding moiety (e.g., a biotin) through a cleavable linker, e.g., a photocleavable, reducing agent cleavable (e.g., a disulphide bridge) or enzymatically cleavable linker. Once the synthesis reaction in the SPE process has been completed, the fully-extended primer, the non-extended primer, and any excess primer is bound to substrate-immobilized binding partner (e.g., streptavidin) and the template strand is removed (e.g., by denaturation and washing). The bound material is then cleaved from the substrate by cleaving the cleavable linker (e.g., by UV illumination if a photo-cleavable linker is used). The resultant non-labeled material can then be blocked using a terminal transferase as described above and subjected to another round of linear amplification in which only fully-extended products are amplified.

In another embodiment of this method, the sorting primer is modified to be resistant to both 5′ and 3′ exonuclease activity (e.g., by incorporating phosphorothioate (PTO) into the sorting primer). Once the synthesis reaction in the SPE process has been completed, the sample is treated with a 5′ exonuclease which will digest template fragments but not the fully-extended primer, the non-extended primer, and any excess primer. The resultant products can then be blocked using a terminal transferase as described above, and subjected to another round of linear amplification in which only fully-extended products are amplified.

Reconstitution of SPIA® Primer Annealing Site (NuGEN Based Amplification Only)

Following denaturation, washing and a streptavidin bead pull-out of the sorting reaction products, the attached fragments may be used in another round of amplification. However, unlike the templates shown in FIGS. 16 and 17 (which are from non-SPIA® reactions), these templates lack the X′ sequence to which the chimeric primer can bind (see FIGS. 11-14, which show the X sequence and chimeric primer binding and extension).

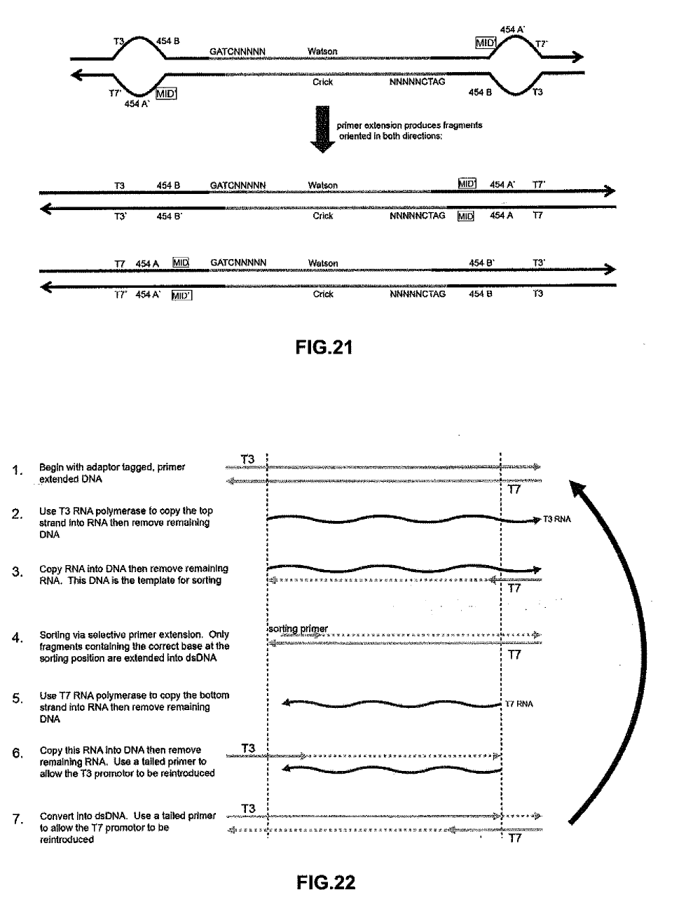

In certain embodiments, the X′ sequence is reconstituted to allow further rounds of amplification. Note that as shown in FIGS. 16 and 17, the unextended sorting primer is present in the reaction. However, if the unextended sorting primer is terminated as shown in FIG. 17, this does not participate in the reactions described below, and thus is not shown in FIGS. 18, 19 and 20.

In certain embodiments, reconstitution of the X′ sequence is achieved using an oligonucleotide containing the XY sequence that is blocked at the 3′ end to prevent extension. The XY oligonucleotide anneals to the sorted fragment (as shown in FIG. 18). Extension of the sorted fragment along the tail of the blocked primer reconstitutes the X′ sequence. In this manner, the sorted template is converted to a viable template for SPIA® reaction described above (after removal of the blocked, unextended XY primer from the template, e.g., by washing the beads in a denaturing solution). It is noted here that for this method of X′ region reconstitution to be used, the terminal transferase blocking reaction described above, and shown in FIG. 17, should take place after reconstitution of the X′ region. If performed prior to reconstitution, the template strand will be blocked from primer extension due the presence of a 3′ terminal di-deoxynucleotide.

One possible drawback of the above reconstitution method is that it involves several additional steps. Therefore, in certain other embodiments, the reconstitution reaction is incorporated directly into the SPIA® reaction.

In certain embodiments, the X′ region is reconstituted as shown in FIG. 19. In step one, an XY chimeric primer binds to the sorted template in the Y′ region. (The primer is chimeric because the X region is RNA and the Y region is DNA.) In step 2, a polymerase having DNA polymerase activity (e.g., a reverse transcriptase) extends the template strand to reconstitute the X′ DNA region of the template while at the same time extending the XY to synthesize a copy of the template. At this point, amplification according to the SPIA® system (as described above) can proceed (see step 3 in FIG. 19 as well as FIGS. 11 to 13 above). It is noted here that for this method of X′ region reconstitution to be used, the terminal transferase blocking reaction described above, and shown in FIG. 17, cannot be employed because the template strand will be blocked from primer extension due the presence of a 3′ terminal di-deoxynucleotide.

In certain embodiments, sequential annealing sites are employed to avoid the need to reconstitute a lost site priming site. Asymmetric adapters can be designed to include multiple sequential primer binding sites. For example, an adapter can be designed to include multiple sequential primer binding sites such that after extension with a biotinylated primer, binding to streptavidin beads, denaturation and bead pull-out (as described in detail above), the resulting template for amplification has four primer binding sites (W′X′Y′ and Z′ as shown in step 1 of FIG. 20). In the first round of amplification using this template, a chimeric primer consisting of W (RNA) and X (DNA) could be used, so that the amplified product would still contain X, Y and Z, but has lost W (FIG. 20, step 2). Subjecting these amplified fragments to sorting (e.g., by sequence specific sorting as described above) produces new templates for amplification (as shown in step 3 of FIG. 20). Amplification of this template would employ a chimeric primer having X (RNA) and Y (DNA) regions. Following the next sorting round, the new template would lack X′, so the amplification primer would include Y (RNA) and Z (DNA) regions.

### RNA-Based Amplification and Sorting of Asymmetrically Tagged Nucleic Acids

Tagging and First Round Replication

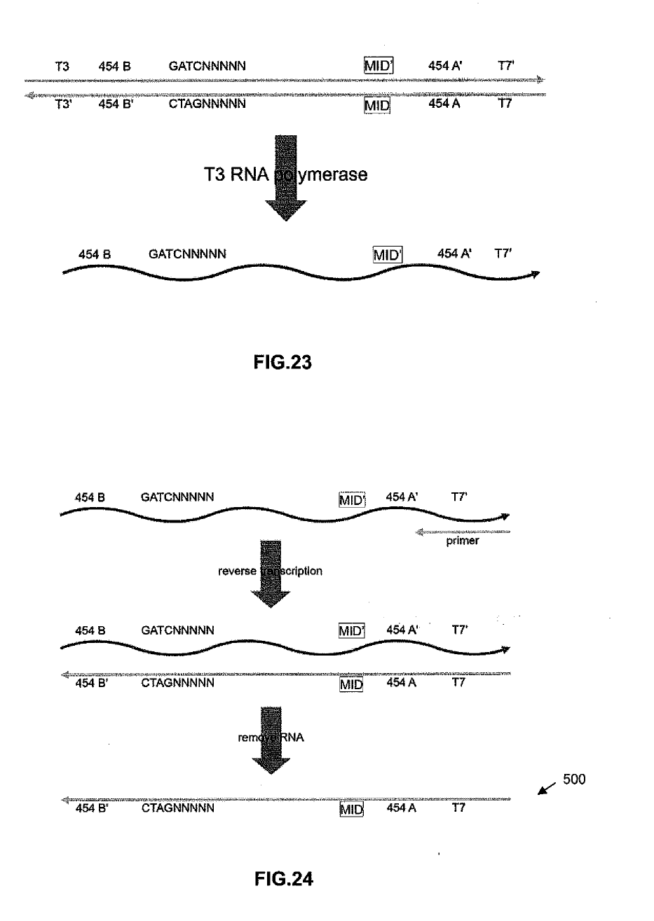

An asymmetrically tagged library is produced in much the same way as described previously, except that the asymmetric adapter includes additional functional sequence elements. The structure of an adapter tagged fragment is shown in FIG. 21, together with the products of the subsequent primer extension reaction to produce asymmetric fragments. The designations T3 and T7 represent the promoter sequences for T3 and T7 RNA polymerases respectively; MID represents the unique identifier sequence; and 454 A and 454 B represent the A and B sequences employed in the Roche 454 sequencing system. In certain embodiments, different sequencing primer binding sites can be employed (e.g., Illumina). The location of the sorting bases (which will be discussed below) is indicated by NNNNN, and the prime symbol (′) refers to a complement sequence, for example, A′ is the complement of A (as described above).

Sorting Tagged and Extended DNA

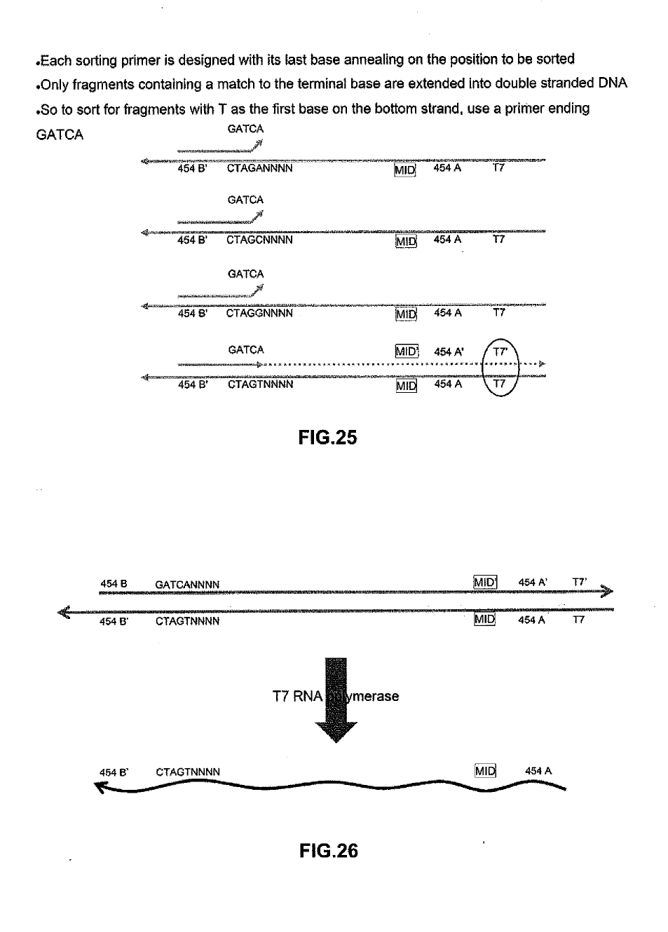

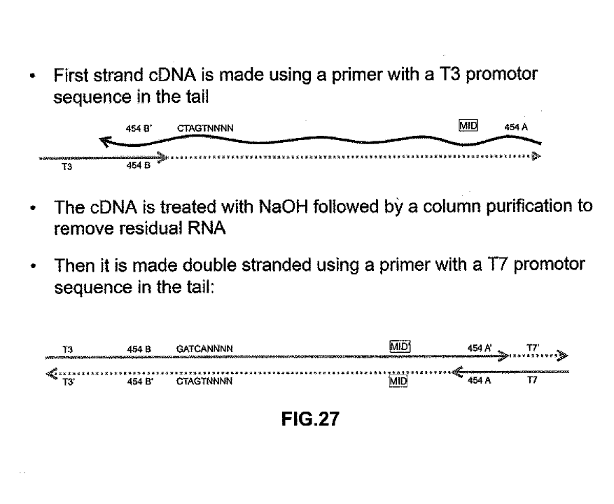

The procedure for sorting tagged and extended DNA into subgenomic pools is summarized in FIG. 22, with details for certain steps shown in FIGS. 23 to 27.

The process shown in FIG. 22 has certain features to note. First, because each step of the process shown in FIG. 22 operates from the opposite end of the fragment to the previous one, only complete (or full-length) fragments can proceed to the next stage. Second, switching between DNA and RNA templates allows unwanted material to be removed between steps. For example, following selective primer extension (step 4 of FIG. 22 and also FIG. 25), only the fully extended fragments (or the desired fragments) are copied into RNA; the rest (or non-desired fragments) are removed with DNase treatment. Third, it is possible to perform a multiple base sort (e.g., a two base sort) in a single step by using a primer that extends multiple bases into the sorting region, thereby reducing the number of cycles required (e.g., by twofold for a two base sorting step).

1. In Vitro Transcription (IVT) with T3 RNA Polymerase

In the first step, the top strand of each fragment is copied into RNA by T3 RNA polymerase (FIG. 23). In the first round, the template strand is the tagged and extended material, while in subsequent rounds it is double stranded sorted DNA produced following reverse transcription on T7 RNA (as described below). Note that the T3 promoter sequence is lost in the T3 RNA copies. Remaining DNA is removed by treatment with a DNase enzyme and the RNA is then purified (e.g., using Qiagen RNeasy columns).

2. Reverse Transcription of T3 RNA to Produce a Template for Sorting

The T3 RNA is then copied back into DNA by reverse transcription to produce single stranded template suitable for input into a sorting reaction (e.g., using a synthesis primer that primes in the T7′ region as shown in FIG. 24). Residual RNA is removed, e.g., by treatment with sodium hydroxide, and the complementary DNA (cDNA) produced is purified, e.g., using a Millipore Microcon column.

3. Sorting by Selective Primer Extension

Sequence-specific sorting of the template produced above can be achieved using a selective primer extension method as described above (see FIG. 15 and its description). In certain embodiments, the template fragments in the sample are sorted into four subgenomic pools (one for each possible nucleotide base) using a specific cognate sorting primer that extends into the sorting region by one base. As noted above, multiple bases may be sorted at a time using sorting primers that extend by more than one base into the sorting region. As depicted in FIG. 25, only fragments having a complementary base to the 3′ nucleotide in the primer (in this case a template having a T to match the terminal A in the primer) are fully extended and gain an active T7 promoter site for the next step.

4. IVT with T7 RNA Polymerase

As indicated above, only the fully extended products will possess a double stranded T7 promoter site following sorting by selective primer extension (SPE). These fully extended products are copied into RNA by T7 RNA polymerase and residual DNA is removed, e.g., by treatment with a DNase enzyme (FIG. 26). The RNA can be purified further (e.g., using a Qiagen RNeasy column). As shown in FIG. 26, the T7 promoter site is lost from the RNA copies.

5. Reconstitution of the T3 and T7 Promoter Sites

In order for the sorting cycle to continue, the lost T3 and T7 promoter sites need to be reconstituted into the fragments. In certain embodiments, this is achieved in two steps (FIG. 27). The first step is to perform a reverse transcription reaction with a primer containing the T3 promoter sequence in the tail (FIG. 27). Residual RNA is destroyed by treatment with sodium hydroxide and the cDNA produced is neutralized before being purified using a Millipore Microcon column. An extension is performed with a primer containing the T7 promoter site in the tail to produce double stranded fragments with the T3 and T7 sequences at each end (FIG. 27). This material is then ready for region of interest (ROI) extraction and subsequent next generation sequencing, or for another round of sorting, starting with the T3 IVT step (step 2, FIG. 22).

### Immortalization and Immortalized Libraries

As noted above, certain polynucleotide samples employed herein or produced during certain process steps, either are, or can be, immortalized. An “immortalized” sample is a sample from which copies can be made without consuming the original sample. For example, an immortalized sample can be used to generate starting material for as many independent assays as needed and, in certain embodiments, can be stored for future use in any desired assay or analysis.

In certain embodiments, an immortalized library is a pooled sample of adapter tagged polynucleotides, where by “pooled” is meant that the polynucleotides in the sample are derived from multiple different samples, e.g., different genomic samples from different individuals. Each polynucleotide in the sample has an attached adapter (which could be a single adapter or two adapters, e.g., asymmetric adapters as described herein) that includes a first common copying initiation site that is positioned to produce an amplified, pooled polynucleotide product (or copies) when placed under appropriate replication conditions (i.e., specific for the first common replication site). By “common copying initiation site” is meant that the same copying initiation site is common to all the adapters on the polynucleotides in the pooled sample regardless of their source. Thus, copying of the polynucleotides from the different sources can be achieved using a single polynucleotide copying condition (e.g., a single nucleic acid synthesis primer or PCR primer pair will generate copies of all polynucleotides in the pooled sample). Each of the polynucleotide products/copies produced will include the polynucleotide with its corresponding MID (and/or complements thereof). It is important to design the copying initiation site(s) such that the coupling of the MID and polynucleotide is maintained, such that in subsequent processing and analysis the source from which a polynucleotide is derived can be accurately discerned, i.e., by MID identification. In certain embodiments, the adapter-attached polynucleotides in the pooled library include a binding moiety (as described above, e.g., biotin).

It is noted here that in certain embodiments, a pooled immortalized library may include only a subset of fragments present in the multiple source samples. For example, an immortalized pooled library may contain polynucleotides that have a common nucleic acid sequence or locus, including but not limited to single nucleotide polymorphisms (SNPs) or other mutations/variations, a specific nucleotide or nucleotide sequence in a sorting region (as detailed above), a hybridization primer binding site (e.g., a nucleic acid synthesis primer binding site or a capture primer binding site), and the like. Moreover, an immortalized pooled library may be used to generate polynucleotide copies of only a subset of the polynucleotides present in the immortalized pooled library, for example by using a sorting primer or a locus/SNP/mutation-specific nucleic acid synthesis primer to initiate nucleic acid synthesis. The identity of the polynucleotide copies generated from an immortalized pooled library is up to the desires of the user and thus no limitation in this regard is intended.

Aspects of the present invention include methods of generating an immortalized pooled polynucleotide sample by combining adapter-attached polynucleotides from multiple samples to produce a pooled sample, where the adapter on each of the adapter-attached polynucleotides includes a first common replication site and a Multiplex Identifier (MID) corresponding to its sample of origin. The first common replication site is positioned to produce a pooled polynucleotide product that includes polynucleotide copies of each adapter-attached polynucleotide in the pooled sample when placed under replication conditions specific for the first common replication site. Each of the polynucleotide copies includes the MID and the polynucleotide (and/or complements thereof), thereby generating an immortalized pooled polynucleotide sample.

In certain embodiments, the adapter-attached polynucleotides include first and second terminal asymmetric adapters. The first terminal asymmetric adapter includes the first common replication site and the second terminal asymmetric adapter may include a second common replication site, where the second common replication site is positioned to produce a pooled polynucleotide product comprising polynucleotide copies of each adapter-attached polynucleotide in the pooled sample when placed under replication conditions specific for the second common replication site. As noted above, each of the polynucleotide copies produced from the second common replication site includes the MID and the polynucleotide (and/or complements thereof).

In certain embodiments, the first and second common replication sites are selected independently from nucleic acid synthesis primer binding sites and nucleotide polymerase binding sites. For example, the first and second common replication sites may represent binding sites for a PCR primer pair. In some cases, the first and second common replication binding sites are opposing T3 and T7 RNA promoter sites, respectively. Combinations of nucleic acid synthesis primer binding sites and nucleotide polymerase binding sites may also be employed in an immortalized pooled library, e.g., one adapter having a nucleic acid synthesis primer binding site and the other adapter having a nucleotide polymerase binding site. As described above, adapter regions may contain other functional domains as desired by the user. For example, an adapter domain may include multiple different common replication sites that find use in generating pooled polynucleotide copies using a variety of replication strategies.

In certain embodiments, the method further includes attaching the combined adapter-attached polynucleotides in the pooled sample to a solid support either non-covalently (e.g., via a binding moiety/binding partner interaction) or covalently. When binding moieties/binding partners are employed, any convenient binding partner pair may be used (described above), where in certain embodiments the binding partner pair is biotin/streptavidin. In certain embodiments, the attaching step includes annealing the combined adapter-attached polynucleotides in the pooled sample to primers attached via their 5′ termini to the solid support followed by extending the annealed primers to generate complements of the asymmetrically tagged polynucleotides. The replicated copies are thus attached to the support (the template strands can be eluted and washed away). The primer may be attached covalently or non-covalently as desired. In certain embodiments, the solid support attached primer is a sorting primer (as described in detail above), where the sorting primer includes at least one sorting nucleotide positioned at a first sorting site in the adapter-attached polynucleotide. In this embodiment, only adapter-attached polynucleotides having nucleotides in the first sorting site complementary to the at least one sorting nucleotide in the sorting primer are extended. The sorting primer may be made resistant to 3′ to 5′ exonuclease digestion. In other embodiments, it is the template strands that are attached to the solid support (e.g., via binding partner or covalently) such that primer extension products are released into the supernatant for use in subsequent analysis. The templates will thus be retained to produce subsequent copies as needed.

Aspects of the present invention include immortalized pooled polynucleotide sample compositions having the features described above. As such, the pooled samples may include combined adapter-attached polynucleotides from multiple samples, where the adapter on each of the adapter-attached polynucleotides includes a first common replication site and a Multiplex Identifier (MID) corresponding to its sample of origin. The first common replication site is positioned to produce a pooled polynucleotide product containing polynucleotide copies of each adapter-attached polynucleotide in the pooled sample when placed under replication conditions specific for the first common replication site. Each of the polynucleotide copies includes the MID and the polynucleotide and/or complements thereof. The immortalized pooled polynucleotide sample can be attached to a solid support.

In certain embodiments, the adapter-attached polynucleotides include first and second terminal asymmetric adapters, and wherein the first terminal asymmetric adapter includes the first common replication site and the second terminal asymmetric adapter includes a second common replication site. As discussed above, the second common replication site is positioned to produce a pooled polynucleotide product having copies of each adapter-attached polynucleotide in the pooled sample when it is placed under replication conditions specific for the second common replication site. Each of the polynucleotide copies will include the MID and the polynucleotide sequence (and/or complements thereof).

The first and second common replication sites can be selected independently from nucleic acid synthesis primer binding sites and nucleotide polymerase binding sites, where in certain cases the first and second common replication sites are opposing T3 and T7 RNA promoter sites, respectively.

It is noted here that many of the pooled asymmetrically tagged samples described above and shown in the figures are amenable to immortalization. Certain exemplary tagged and pooled polynucleotide samples shown in FIGS. 7 and 10 (indicated by 500) are immortalized pooled samples. However, other polynucleotide samples not indicated may also be immortalized, and thus no limitation in this regard is intended.

### Kits, Systems and Service Offerings

Also provided by the subject invention are kits and systems for practicing the asymmetric tagging, amplification and sorting methods, as described above. For example, kits or systems according to the subject invention may include components and reagents for producing asymmetrically tagged nucleic acid fragments, e.g., asymmetric adapters, restriction enzymes, ligases, polymerases, reagents for “polishing” the ends of nucleic acid fragments to create adapter-compatible ends (e.g., nucleotides, polymerases etc.), reagents for performing at least a first round of replication of the asymmetrically tagged fragments after adapter ligation (e.g., nucleotides, polymerases, primers, etc.), binding moiety tagged reagents (e.g., asymmetric adapters and synthesis primers), and substrates (e.g., beads, pins, plates, etc.) with immobilized binding partner for the binding moiety (e.g., for isolating binding moiety tagged nucleic acids/oligonucleotides). Kits or systems according to the subject invention may include components and reagents for performing any one or more of the steps for producing single stranded asymmetrically tagged copies suitable for sorting, e.g., DNA and/or RNA polymerases, synthesis primers, nucleotides, etc. Kits and systems according to the subject invention may include components and reagents for performing any one or more of the steps detailed above for sorting by selective primer extension (SPE), e.g., one or more sorting primer (e.g., multiple indexed sorting primers for sorting multiple sequential bases in a sorting region of a tagged nucleic acid fragment), DNA and/or RNA polymerases, nucleases, exonucleases, terminal transferases, nucleotides, synthesis terminating nucleotides (e.g., ddNTPs), binding moiety tagged reagents (e.g., sorting primers) and substrates (e.g., beads, pins, plates, etc.) with immobilized binding partner for the binding moiety.

The various components of the kits may be present in separate containers or certain compatible components may be precombined into a single container, as desired.

The subject systems and kits may also include one or more other reagents or components for preparing or processing a nucleic acid sample according to the subject methods. These may include one or more matrices, solvents, sample preparation reagents, buffers, desalting reagents, enzymatic reagents, denaturing reagents, where calibration standards such as positive and negative controls may be provided as well. As such, the kits may include one or more containers such as vials or bottles, with each container containing a separate component for carrying out a sample processing or preparing step and/or for carrying out one or more steps of an SPE assay according to the present invention.

In addition to the above-mentioned components and reagents, the subject kits typically further include instructions for using the components of the kit to practice the subject methods, e.g., to sort asymmetrically tagged nucleic acid fragments according to aspects of the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control samples and reagents, e.g., two or more control samples for use in testing the kit.

In certain embodiments, aspects of the present invention include providing the nucleic acid sorting methods described above, or the product generated at any step of the methods, as a service to a client. In certain embodiments, the service provider may supply to a client one or more pre-tagged, amplified and sorted samples in response to a request from the client. For example, a client may request all or a subset of pre-sorted samples derived from a specific nucleic acid source (e.g., a genome, e.g., from a human, bacteria, yeast, etc.), where the fragments have been sorted in a specified manner (e.g., sorted based on the identity of five nucleotides in a sorting region). In certain other embodiments, a client may provide one or more nucleic acid sample to the service provider and request that this sample (or samples) be sorted into subsets based on the identity of one or more bases in a sorting region. As such, aspects of the present invention include receiving a nucleic acid sorting request from a client and providing to the client one or more samples based on the sorting request. A sorting request can include any type of information relevant to sorting a nucleic acid sample according to the methods described above, including, but not limited to: a nucleic acid sample to be sorted, a species name, the number of bases to be sorted, a sequence of the sorting site to be sorted, a downstream assay to be performed using the sorted sample, and an asymmetric adapter parameter (e.g., functional domains, ligation site, size, etc.).

### Utility

The sorting methods, kits, systems and services described herein enables one to amplify and sort an asymmetrically tagged nucleic acid sample or pooled samples based on the identity of one or more nucleotides in a sorting region. This sorting process allows the complexity of a starting nucleic acid sample to be reduced in a controlled and reproducible manner, facilitating downstream manipulation (e.g., ROI extraction, sequence analysis, culling, etc.). As such, the subject invention can be integrated into a variety of nucleic acid analyses currently being performed (e.g., high throughput sequencing assays) as well as provide a catalyst for the development of novel assays that rely on the efficient and systematic sorting of nucleic acid fragments from in complex samples. Therefore, no limitation with regard to the types of assays to which the subject invention may be applied is intended.

### EXAMPLES

### Example I

The Example described below shows the amplification and subsequent sorting of an initial asymmetric adapter ligated sample into separate populations by sequence specific sorting. Specifically, the Example below describes sorting a population of asymmetrically tagged nucleic acid fragments at five consecutive sorting positions in five separate cycles of sorting. The source for the nucleic acid fragments processed was genomic DNA from E. coli. The sorting method employed in this Example is the one summarised in FIG. 22 (described above). All steps for completing a single round of sequence specific sorting are described in detail below, with a separate heading for each step (numbers in the headings correspond to the stages shown in FIG. 22).

### Abbreviations:

### ABI=Applied Biosystems

DTT=dithiothreitol

### ET SSB=Extreme Thermostable Single-Stranded Binding Protein (NEB)

IVT=in vitro transcription
LNA=locked nucleic acid

### NEB=New England Biolabs

NaOH=sodium hydroxide
PTO=phosphorothioate
ROI=region of interest
RT=reverse transcription
SSII=SuperScript II reverse transcriptase (Invitrogen)

### Step 1. Asymmetrically Tagged Library

An asymmetrically tagged library was prepared from E. coli genomic DNA. The resultant adapter ligated and primer extended nucleic acid library had the domain structure shown in FIG. 21. The DNA concentration of this library was approximately 20 ng/μL.

Step 2. IVT with T3 RNA Polymerase and Removal of DNA Template Followed by RNA Purification

In this step, the asymmetrically tagged library produced in step 1 was subjected to in vitro transcription (IVT) using T3 RNA polymerase. In the first round, the template was the tagged and extended material (as depicted in FIG. 23). In subsequent rounds, the template was double stranded sorted DNA produced following reverse transcription of RNA produced by T7 RNA polymerase (see steps 6 and 7 of FIG. 22, as described below). Reaction conditions for T3-based IVT reaction were as follows:

| | Stock | | Reaction | | |
|---|---|---|---|---|---|
| Reagent | concentration | | concentration | | Volume (μL) |
| water | N/A | | N/A | | 44.8 |
| IVTP050208 buffer* | 10× | | 1× | | 8.0 |
| rNTPs** | 25 | mM | 1 | mM | 3.2 |
| T3 RNA Polymerase- | 20 | U/μL | 1 | U/μL | 4.0 |
| P1us ™ Enzyme | | | | | |
| Mix (ABI) | | | | | |
| Tagged and extended | | | | | |
| DNA or sorted | ~20 | ng/μL | ~400 | ng | 20.0 |

dsDNA

*350 mM Tris-HCl (pH 8.0), 55 mM MgCl₂, 100 mM DTT, 20 mM Spermidine, 0.5% Tween20.
**rNTPs = mixture of all four ribonucleotide triphosphates (ATP, GTP, CTP, UTP)

The reaction volume was 80 μL; the reaction was incubated at 37° C. for 4 hours. To remove residual DNA, the sample was treated with 4 μL of TURBO™ DNase (ABI, 2 U/μL) and incubating at 37° C. for 15 min. RNA purification was carried out using a Qiagen RNeasy Mini kit. The reaction volume was made up to 100 μL with water then 350 μL of buffer RLT (included in the kit) was added and the sample was mixed well by pipetting. 250 μL ethanol was added and mixed by pipetting again before immediately transferring to a column and spinning at >10,000 rpm for 15 seconds. The supernatant was discarded and 500 μL buffer RPE (included in the kit) was added. The sample was centrifuged at >10,000 rpm for 15 seconds and the supernatant discarded. Another 500 μL buffer RPE was added and the sample spun for 2 minutes at >10,000 rpm. The supernatant was discarded and the column transferred to a fresh collection tube and centrifuged at top speed for 1 minute. The column was then transferred to a collection tube, 40 μL of water was added, and the column spun at >10,000 rpm for 1 minute.

The RNA eluted was quantified using a NanoDrop spectrophotometer. In the first and subsequent rounds, the concentration obtained was in the range of 200-600 ng/μL in a volume of just under 40 μL.

Step 3. Reverse Transcription (RT) on T3 IVT RNA to Produce a Template for Sorting; Clean-Up of T3 cDNA

Reverse Transcription of the T3 IVT RNA was carried out as shown in FIG. 24. Primer annealing was carried out as follows:

| | Stock | | Reaction | | Volume |
|---|---|---|---|---|---|
| Reagent | concentration | | concentration | | (μL) |
| water | N/A | | N/A | | variable |
| primer | 10 | μM | 120 | pmol per reaction | 12.0 |
| SUPERase | 20 | U/μL | 0.2 | U/μL | 0.24 |

| In ™ RNase | | | | |
|---|---|---|---|---|
| Inhibitor (ABI) | | | | |
| T3 RNA | variable | 4000 | ng per reaction | variable |

The reaction volume was 23.8 μL; the sample was incubated at 65° C. for 5 min, then cooled on ice. After cooling, the RT reaction was carried out by adding the following:

| | Stock | | Final reaction | | |
|---|---|---|---|---|---|
| Reagent | concentration | | concentration | | Volume (μL) |
| First-strand buffer | 5× | | 1× | | 8.0 |
| (Invitrogen) | | | | | |
| DTT | 100 | mM | 10 | mM | 4.0 |
| dNTPs | 25 | mM | 1 | mM | 1.6 |
| T4 Gene 32 Protein | 10 | mg/mL | 0.05 | mg/mL | 0.2 |
| (NEB) | | | | | |
| SUPERase In ™ RNase | 20 | U/μL | 0.2 | U/μL (plus 0.2 U/μL from above) | 0.4 |

Inhibitor (ABI)

The Reaction volume was 38 μL; the sample was incubated at room temperature for 5 min, after which the following reagents were added:

| | Stock | Reaction | |
|---|---|---|---|
| Reagent | concentration | concentration | Volume (μL) |
| SuperScript ™ II Reverse | 200 U/μL | 10 U/μL | 2μL |
| Transcriptase (Invitrogen) | | | |

The reaction volume was 40 μL; the reaction was incubated at 45° C. for 1 hour. After the RT reaction was completed, RNA was degraded by treatment with 0.1 M NaOH. Specifically, 1 μL, of 5 M NaOH and 9 μL, water were mixed and then added to the reaction to give a 50 μL, volume which was then incubated at 37° C. for 10 min.

The remaining cDNA was purified using a Microcon YM-50 column (Millipore). Briefly, the column was prepared by adding 500 μL, water and marking the level with a permanent marker. The column was centrifuged at 14,000 rcf for 4 min and the supernatant discarded. 100 μL, of 1 M Tris-HCl (pH 7) was added to the column, together with the NaOH treated RT products, and water was added up to the marked line. The column was centrifuged at 14,000 rcf for 4 min. The supernatant was discarded and the sample washed by filling up to the marked line with water and centrifuging again at 14,000 rcf for 4 min. This wash step was repeated twice more.

The column was then inverted and placed in a clean tube and spun at 11,000 rcf for 1 minute (the volume of liquid eluted in each round was generally between 30 and 50 μL). The sample was quantified using a NanoDrop spectrophotometer. The concentration obtained in each round was in the range of 20-80 ng/μL.

### Step 4. Sorting by Selective Primer Extension (SPE-1 Base Sort)

The sorting procedure employed was designed to perform a five base sort in five successive rounds, each round sorting for a single base. The sequence of bases sorted for the first four rounds is: T T C T (where the T T C T sequence is the sequence immediately after the GATC sequence shown on the top strand of the DNA in FIG. 23, i.e., the four “Ns”). After the first four rounds were completed, the sample was sorted into four separate subgenomic pools, each subgenomic pool having a different base at the fifth position (i.e., the fifth “N” after the GATC sequence). Therefore, the four pools will have the following sequences after the GATC: T T C T G; T T C T A; T T C T T; T T C T C (where the underlined base is the one sorted in the 5^{th} round.

For the first round of sorting, the sorting primer ended with GATCT, where the terminal “T” is complementary to the base to be sorted on the template strand, in this case an “A”. Because the enzyme used for the extension reaction, Vent DNA polymerase, exhibits 3′ to 5′ exonuclease activity, the sorting primer employed was protected from digestion by this enzymatic activity by including a phosphorothioate (PTO) modification at the 3′ end. This modification leads to “stalling” of the enzyme at a mismatched site, as it is unable to remove the mismatched base (the terminal 3′ base). Thus, only matched fragments are fully extended and gain an active T7 promoter site for the next step (see FIG. 25).

The sorting reaction was set up as follows:

| | Stock | | Reaction | | Volume |
|---|---|---|---|---|---|
| Reagent | concentration | | concentration | | (μL) |
| Thermopol buffer (NEB) | 10× | | 1× | | 2.0 |
| Sorting primer* | 2.5 | μM | 0.4 | μM | 3.2 |
| dNTPs | 10 | mM | 0.1 | mM | 0.2 |
| Vent DNA polymerase (NEB) | 2 | U/μL | 0.02 | U/μL | 0.2 |
| ET SSB (NEB)† | 1 | mg/mL | 4 | ng/μL | 0.08 |

| | | | |
|---|---|---|---|
| T3 cDNA | variable | variable | 14.32 |

*If the primer used has a 3′ phosphorothioate modification, it must be pre-digested with T4 DNA polymerase.
See below for more details.
†This can be diluted to make pipetting easier.

The reaction volume was 20 μL; the reaction was incubated at 95° C. for 5 min, 70° C. for 30 sec, and 72° C. for 10 min.

### Pre-Treatment of Phosphorothioate Modified Primers

This is necessary to remove the isomer that vent is able to digest.

| | Stock | | Reaction | | Volume |
|---|---|---|---|---|---|
| Reagent | concentration | | concentration | | (μL) |
| water | | | | | 76.0 |
| NEBuffer 2 (NEB) | 10× | | 1× | | 10.0 |
| PTO modified sorting primer | 100 | μM | 10 | μM | 10.0 |
| T4 DNA polymerase (NEB) | 3 | U/μL | 0.12 | U/μL | 4.0 |

The reaction volume was 100 μL; the sample was incubated at 37° C. overnight, and then 75° C. for 20 min. This treatment results in an approximate primer concentration of 2.5 μM.

Each subsequent sorting reaction was performed as above except that the sorting primer was indexed to the next sorting base. For example, in the second round of sorting, the primer ended with the sequence GATCTT, where the penultimate T pairs with the base sorted in the first sorting round (i.e., for A in the template strand) and the terminal T (underlined) is at the new, indexed sorting position.

While not done in this specific Example, it is also possible to sort two bases per cycle by using a primer extending two bases into the sorting region (as detailed in previous sections).

Step 5. IVT with T7 RNA Polymerase, Removal of DNA and RNA Purification

As noted above, only the fully extended products (i.e., products having the desired base at the sorting position) will possess a double stranded T7 promoter site following SPE. Therefore, only these fully extended products can serve as a template for T7 RNA polymerase.

Sorted T3 cDNA was subjected to T7 IVT as follows (see FIG. 26 for diagram of reaction):

| | Stock | | Reaction | | Volume |
|---|---|---|---|---|---|
| Reagent | concentration | | concentration | | (μL) |
| water | | | | | 44.8 |
| IVTP050208 buffer | 10× | | 1× | | 8.0 |
| rNTPs | 25 | mM | 1 | mM | 3.2 |
| T7 RNA Polymerase-Plus ™ | 20 | U/μL | 1 | U/μL | 4.0 |
| Enzyme Mix (ABI) | | | | | |
| Sorted T3 cDNA | ~5-15 | ng/μL | ~1.2-3.6 | ng/μL | 20.0 |

The reaction volume was 80 μL; the reaction was incubated at 37° C. for 4 hours.

After this reaction, any remaining DNA was removed by adding 4 μL, of TURBO™ DNase (ABI, 2 U/μL) and incubating at 37° C. for 15 minutes.

RNA purification was carried out using a Qiagen RNeasy Mini kit according to the manufacturer's instructions (see section 2, above, for further details). The RNA eluted was quantified using a NanoDrop spectrophotometer. The resulting concentration in each round of sorting was in the range of 50-200 ng/μL.

Step 6. RT on T7 RNA and Clean-Up of T7 cDNA

The T7 RNA produced in step 5 was then subjected to a reverse transcription (RT) reaction (see FIG. 27). In order to allow subsequent rounds of sorting, the T3 promoter region lost during prior manipulations needed to be reconstituted. To accomplish this, the DNA primer employed in the RT reaction had a 5′ domain (or “tail”) that included a T3 promoter sequence (in addition to the region complementary to the 3′ end of the T7 RNA). It is noted here that the T3 tail is only necessary if another round of sorting is to be performed. If the sorted sample is to be submitted, e.g., for region of interest extraction or for 454 sequencing, this T3 tail is not required.

The reaction conditions were as follows:

### Primer Annealing:

| | Stock | | Reaction | | Volume |
|---|---|---|---|---|---|
| Reagent | concentration | | concentration | | (μL) |
| Tailed primer | 10 | μM | 50 | pmol total | 5.0 |
| SUPERase In ™ RNase | 20 | U/μL | 0.2 | U/μL | 0.24 |

| Inhibitor (ABI) | | | | |
|---|---|---|---|---|
| T7 RNA | (variable) | 4000 | ng total | variable |

The reaction volume was 24 μL; the sample was incubated at 65° C. for 5 min, then cooled on ice. After cooling, the following RT reagents were added:

| | Stock | | Final reaction | | |
|---|---|---|---|---|---|
| Reagent | concentration | | concentration | | Volume (μL) |
| First-strand buffer | 5× | | 1× | | 8.0 |
| (Invitrogen) | | | | | |
| DTT | 100 | mM | 10 | mM | 4.0 |
| dNTPs | 25 | mM | 1 | mM | 1.6 |
| T4 Gene 32 Protein | 10 | mg/mL | 0.05 | mg/mL | 0.2 |
| (NEB) | | | | | |
| SUPERase In ™ RNase | 20 | U/μL | 0.2 | U/μL (plus 0.2 U/μL from above) | 0.4 |

Inhibitor (ABI)

The reaction volume was 38 μL; the sample was incubated at room temperature for 5 min. After incubation, the following was added:

| | Stock | Reaction | |
|---|---|---|---|
| Reagent | concentration | concentration | Volume (μL) |
| SuperScript ™ II Reverse | 200 U/μL | 2 U/μL | 2 μL |
| Transcriptase (Invitrogen) | | | |

The reaction volume was 40 μL; the sample was incubated at 45° C. for 1 hour. RNA was removed from the sample by treatment with 0.1 M NaOH followed by a Microcon column purification as detailed in step 3 above. The cDNA concentration achieved in different rounds of this step was generally in the range 30-100 ng/μL.

Step 7. Convert T7 cDNA to dsDNA

Following the RT reaction, the material was ready for to be processed according to its location in the workflow. When the cDNA was to be subjected to another round of sorting, it was used as a template for DNA synthesis to produce a suitable dsDNA product. Specifically, the single stranded cDNA was subjected to a DNA synthesis reaction designed to reconstitute the T7 promoter sequence lost in prior manipulations. To accomplish this, the synthesis primer used in this reaction was tailed with a T7 promoter sequence (see FIG. 27).

The reaction was performed as follows:

| | Stock | | Reaction | | |
|---|---|---|---|---|---|
| Reagent | concentration | | concentration | | Volume (μL) |
| Thermopol buffer | 10× | | 1× | | 2.0 |
| (NEB) | | | | | |
| dNTPs | 10 | mM | 400 | μM | 0.8 |
| Tailed primer | 10 | μM | 0.5 | μM | 1.0 |
| Vent DNA | 2 | U/μL | 0.02 | U/μL | 0.2 |

| | | | |
|---|---|---|---|
| polymerase (NEB) | | | |
| T7 cDNA | variable | variable | 16.0 |

The reaction volume was 20 μL; the reaction was incubated at 95° C. for 5 min, 60° C. for 30 sec, and 72° C. for 10 min.

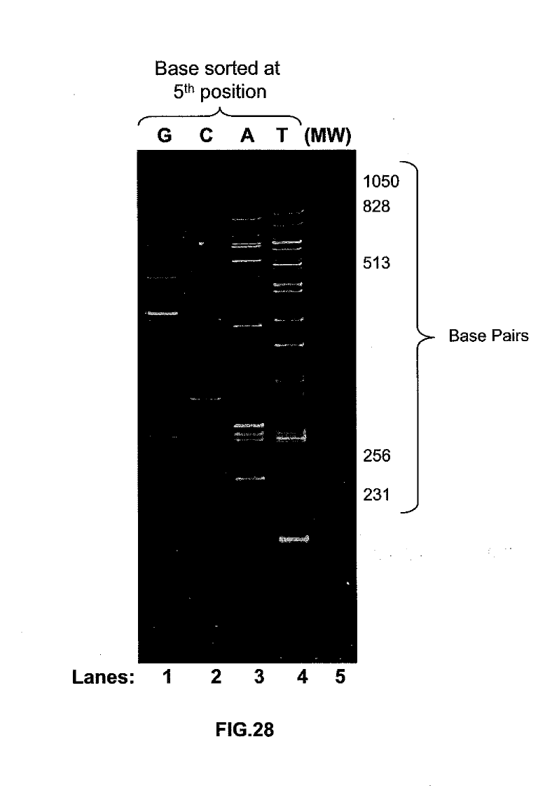

As shown in FIG. 27, dsDNA produced in this step includes both the T3 and T7 promoter sequences, making it suitable for subsequent rounds of sorting. In the present Example, the starting fragments were sorted in sequential sorting rounds as detailed above for T, T, C and T at the first four sorting positions (where the T, T, C and T represent the four N bases following the GATC in the top strand shown in FIG. 23). In the last round of sorting (the 5^{th} round), the sample was split into four separate samples, each of which was sorted for a different deoxy-nucleotide base (i.e., G, C, A or T). In the last T7 RT step of the 5^{th} base sort, a fluorescently labelled primer was used to allow the resultant products to be visualised on a gel (shown in FIG. 28). In FIG. 28, lanes 1, 2, 3 and 4 represent 5^{th} base sorts for G, C, A and T, respectively. Lane 5 is a size marker. As is evident from this gel, each sorted sample includes a unique set of fragments.

Sequence analysis of both the G and C pools analyzed in FIG. 28 (lanes 1 and 2, respectively) was performed using the Roche 454 system. This sequence analysis revealed that 88% of the fragments sorted into the G pool had the sequence T T C T G in the sorting region and 77% of the fragments sorted into the C pool had T T C T C in the sorting region, demonstrating the effectiveness of the five cycles of sorting in reducing the complexity of the starting sample in a sequence-specific manner.

No Bias in MID Representation in Sorted Polynucleotide Samples

In order to determine the effect of the sorting process on the representation of polynucleotides having a different MID (i.e., derived from different source samples), MID representation analysis was performed after sorting reactions (as described above). In this case however, the sorting base was N, so that the copying mechanism could be examined independently of the sorting reaction in terms of MID biases (termed n-sort). The starting material was a single source sample tagged with a degenerate MID sequence, thus creating 81 separate 4 base MIDs. After each n-sort, the sample was sequenced using 454 sequencing to determine the relative amounts of each of the MIDs in the sample. The input (before n-sorting) sample was also sequenced to determine the ‘starting’ MID representation, which accounts for the synthesis bias during degenerate oligonucleotide synthesis. For each of the sequenced samples including the input, the relative number of reads for each MID was calculated with respect to the total number of reads in the sample. These values were used to calculate a Log (base 2) ratio to the input for each of the 81 MIDs in each of the n-sorted samples.

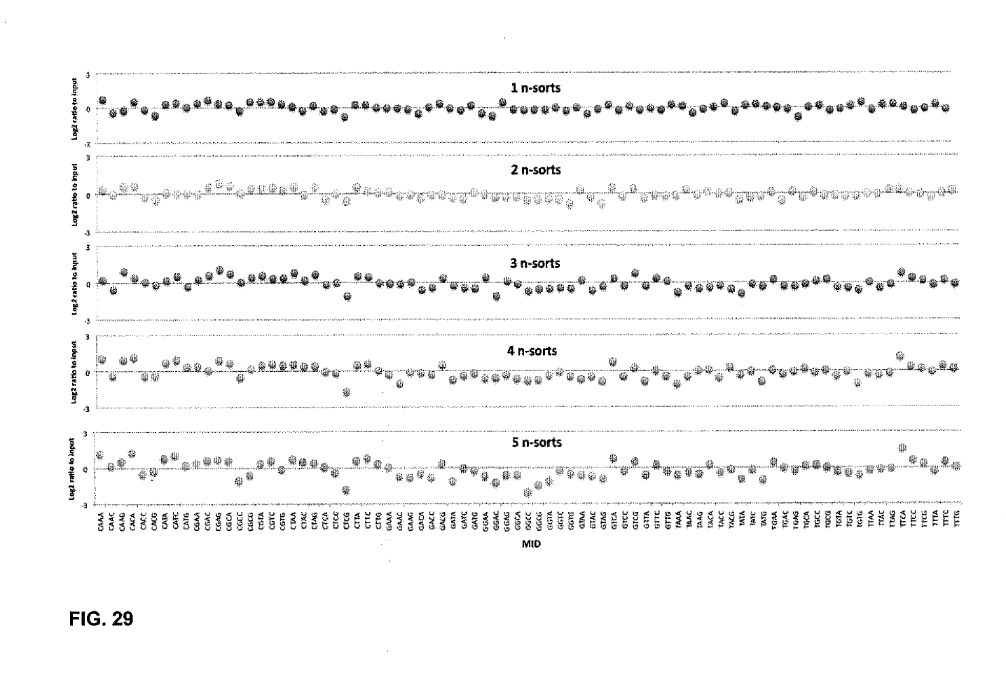

FIG. 29 shows the results of the MID analysis above. The log (base 2) ratio of each n-sorted MID-tagged polynucleotide to input MID-tagged polynucleotide for each MID is shown (Y axis) after each of the 5 n-sorting cycles (5 panels labeled 1 n-sort, 2 n-sort, 3 n-sort, 4 n-sort, and 5 n-sort). The four-nucleotide sequence of each of the 81 MIDs is shown on the X axis.

As is clear from FIG. 29, the sorting process does not lead to significant under- or over-representation of any of the MID tagged polynucleotides. Indeed, MID representation is very consistent from the 1^{st} sort through to the 5^{th} sort, demonstrating the remarkable consistency and completeness of each sorting step. As such, the various steps of the sorting process detailed herein do not result in any significant representational bias of the polynucleotides in the sample (MID bias), making it very useful in the processing and analysis of pooled polynucleotide samples.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1-16. (canceled)

17. A method of sorting a mixture of asymmetrically tagged nucleic acid fragments comprising:
a) producing a single stranded copy of each asymmetrically tagged nucleic acid fragment in the mixture, wherein each asymmetrically tagged nucleic acid fragment comprises:
a first and a second nucleic acid tag on opposite ends of the nucleic acid fragment,
wherein the first and the second nucleic acid tag do not have identical nucleic acid sequences; and
a sorting region in the nucleic acid fragment adjacent to the first tag;
wherein at least a portion of the first nucleic acid tag is present at the 3′ end of the single stranded template and at least portion of the second tag is present at the 5′ end of the single stranded template;
b) annealing a sorting primer to the single stranded template, wherein the sorting primer comprises at least one sorting nucleotide at its 3′ end, wherein the at least one sorting nucleotide is positioned at a first sorting site in the sorting region of the single stranded template;
c) subjecting the sorting primer-annealed single stranded templates to nucleic acid synthesis conditions, wherein only nucleic acid fragments having nucleotides in the first sorting site complementary to the at least one sorting nucleotide in the sorting primer are extended to produce synthesis products; and
d) replicating the synthesis products using a region in the second tag to produce a sorted sample.

18. The method of claim 17, wherein the single stranded templates in step a) are single stranded DNA copies.

19. The method of claim 18, wherein the single stranded DNA copies are produced by a linear thermocycling amplification process using a primer that anneals in the second tag and a thermostable polymerase.

20. The method of claim 18, wherein the single stranded DNA copies are produced from a single stranded RNA copy of the asymmetrically tagged nucleic acid fragments by a reverse transcription reaction using a primer that anneals in the second tag and a reverse transcriptase.

21. The method of claim 20, wherein the single stranded RNA copies are produced by an RNA polymerase from a cognate RNA polymerase promoter site present in the first tag.

22. The method of claim 17, wherein a proofreading polymerase is employed in the nucleic acid synthesis of step c) and the sorting primer is modified to be resistant to 3′ to 5′ enzymatic degradation.

23. The method of claim 22, wherein modification of the sorting primer is selected from the group consisting of: phosphorothioate modification (PTO) and locked nucleic acid modification (LNA).

24. The method of claim 17, wherein the method further comprises isolating the synthesis products of step c) prior to replicating step d).

25. The method of claim 24, wherein the sorting primer comprises a binding moiety and the isolating step comprises contacting the synthesis products to substrate-immobilized binding partners for the binding moiety and removal of the single stranded template.

26. The method of claim 25, wherein the binding moiety is linked to the sorting primer via a cleavable linker and the isolating step further comprises cleaving the binding moiety from the sorting primer.

27. The method of claim 25, wherein the sorting primer is resistant to 5′ to 3′ exonuclease digestion and the isolating step comprises contacting the sample with a 5′ to 3′ exonuclease.

28. The method of claim 24, wherein the isolating step comprises synthesizing an RNA copy of the fully extended template from an RNA polymerase promoter present in the second tag followed by digestion of the template DNA.

29. The method of claim 28, wherein the replicating step comprises synthesizing cDNA from the RNA copy of the fully extended template using reverse transcriptase (RT).

30. The method of claim 29, wherein the replicating step further comprises producing a double-stranded DNA from the cDNA.

31. The method of claim 30, wherein at least a portion of the first and second adapters are reconstituted in the dsDNA.

32. The method of claim 31, wherein the method further comprises subjecting the replicated fragments to another round of sorting using an indexed sorting primer designed to for a second sorting site in the sorting region.

33. The method of claim 17, wherein the first adapter comprises a T3 RNA promoter and the second adapter comprises a T7 RNA promoter and a Multiplex Identifier (MID).

34. The method of claim 33, wherein the first and second adapter further comprise sequencing primer binding sites.

35-36. (canceled)
